(19) United States
(12) Patent Application Publication
(10) Pub. No.: US 2026/0072044 A1
(43) Pub. Date: Mar. 12, 2026

(54) Pulse Stable Tracer Methods for Detection of Short-Chain Fatty Acids

(71) Applicants: Nicolaas E. Deutz, College Station, TX (US); Marielle P. Engelen, College Station, TX (US); Gabrielle A. Ten Have, Montgomery, TX (US); John J. Thaden, College Station, TX (US)

(72) Inventors: Nicolaas E. Deutz, College Station, TX (US); Marielle P. Engelen, College Station, TX (US); Gabrielle A. Ten Have, Montgomery, TX (US); John J. Thaden, College Station, TX (US)

(73) Assignee: The Texas A&M University System, College Station, TX (US)

(21) Appl. No.: 19/391,381

(22) Filed: Nov. 17, 2025

Related U.S. Application Data
(63) Continuation-in-part of application No. 16/968,812, filed on Aug. 10, 2020, now Pat. No. 12,474,352.

(51) Int. Cl. G01N 33/68; A61K 31/733; G01N 33/58

(52) U.S. Cl. CPC G01N 33/6896; A61K 31/733; G01N 33/58; G01N 2458/15; G01N 2800/122; G01N 2800/28; G01N 2800/7085

(57) ABSTRACT

Provided herein are methods for diagnosing a pulmonary disease, for example chronic obstructive pulmonary disorder, in a subject and for diagnosing a neurological disorder, for example, autism in a subject. Also provided is a method for increasing short chain fatty acid production in an older subject. Generally, the methods utilize stable isotope labeled, for example, ¹³C-labeled, short chain fatty acid from which metabolic rates and concentrations are calculated to confirm a diagnosis or increase in the short chain fatty acid production.

### CROSS-REFERENCE TO RELATED APPLICATIONS

This continuation-in-part application claims benefit of priority under 35 U.S.C. § 120 of pending application U.S. Ser. No. 16/968,812, filed Aug. 10, 2020, which is a national stage application under 35 U.S.C. § 371 of international application PCT/US2019/017371, filed Feb. 8, 2019, now abandoned, which claims benefit of priority under 35 U.S.C. § 119 (e) of provisional application U.S. Ser. No. 62/628,376, filed Feb. 9, 2018, now abandoned, the entirety of all of which is hereby incorporated by reference.

### BACKGROUND OF THE INVENTION

### Field of the Invention

The present invention generally relates to the field of nutritional biochemistry. More specifically, the present invention relates to diagnostic methods to assess the metabolism of colonic short-chain fatty acids using stable isotope labeled short-chain fatty acid tracers and to diagnose underproduction of short-chain fatty acids.

### Description of the Related Art

Short-chain fatty acids (SCFAs), the end products of fermentation of dietary fibers by the anaerobic intestinal microbiota are known to exert multiple beneficial effects on body health. Short-chain fatty acids might play a key role in the prevention and treatment of the metabolic syndrome, bowel disorders, and certain types of cancer. In clinical studies, short-chain fatty acid administration positively influenced the treatment of ulcerative colitis, Crohn's disease, and antibiotic-associated diarrhea.

Short-chain fatty acids also appear to have anti-inflammatory and immune modulating effects. In a mouse model, a diet rich in whey proteins attenuated chronic obstructive pulmonary disease (COPD) through suppression of respiratory inflammation, which correlated with high colonic short-chain fatty acids levels. Higher short-chain fatty acids levels have also been detected in the stool of children with autism. Moreover, gastrointestinal diseases and symptoms are significantly prevalent in individuals with COPD and autism compared to those with other diseases/disorders, suggesting a likely relationship of these to increased colonic Short-chain fatty acid. Thus, imbalances in Short-chain fatty acid production have multifaceted effects in vivo thereby impacting body health.

Short-chain fatty acids are straight or branched-chain fatty acids produced by the intestinal microbiota in the large intestine (colon) mainly through fermentation of undigested carbohydrates (soluble fiber), but also through degradation of dietary and endogenous proteins. Acetate (C2), propionate (C3), and butyrate (C4) are the most common short-chain fatty acids in the colon. About 95% of the short-chain fatty acids absorbed by colonocytes and only 5% is excreted with the feces. Colonic epithelial cells oxidize short-chain fatty acids to ketone bodies and CO₂ to about 10% of the daily caloric requirement of humans. Short-chain fatty acid are not only the main energy source of colonocytes, but also contribute to epithelial maintenance, barrier function, and reduction of oxidative stress induced DNA damage. While the gut is primarily responsible for releasing short-chain fatty acids into the circulation, the liver is the main disposal organ for circulating short-chain fatty acid. Thus, the combined action of the colon and the liver keep plasma short-chain fatty acids at low levels. However, short-chain fatty acid levels in the portal and peripheral blood are known to differ substantially between individuals in relation to colonic variations in the microbiota composition and diet.

A clear understanding of the role of short-chain fatty acids on human health require quantitative data on short-chain fatty acid production and the impact this has on host metabolism. Current methods assess in situ production of short-chain fatty acids by measuring their content in feces. Alternative methods use infusion of short-chain fatty acid directly into the colon, or a very unreliable method of primed constant infusion protocol to measure its dilution in plasma. These methodologies however do not accurately represent short-chain fatty acid production in more proximal regions of the colon because colonocytes absorb more than 95% of short-chain fatty acids. Further, since short-chain fatty acids are metabolized by the colon and liver, it becomes difficult to accurately estimate actual short-chain fatty acid production by measurements of the concentration in fecal or plasma samples using current methods.

Nutritional guidelines suggest that the U.S. population needs to consume more fiber in their diet to stimulate the large intestine production of short chain fatty acids. Reduced short chain fatty acid production in the colon relates to a change of the bacterial composition in the large intestine (gut microbiome). By providing more soluble fiber, the gut microbiome is expected to change towards more short chain fatty acid producing bacteria. Chronic diseases like chronic obstructive pulmonary disease and neurological disorders like autism are characterized by gut dysfunction possibly related to a reduced short chain fatty acid production. The relationship between short chain fatty acid and these maladies has however never been proven in humans.

Thus, there is a recognized need in the art for a method of accurately and distinguishably detecting short-chain fatty acid production. The present invention fulfills this long-standing need and desire in the art.

### SUMMARY OF THE INVENTION

The present invention is directed to method for diagnosing chronic obstructive pulmonary disease in a subject. In this method, a metabolic rate of at least one stable-isotope labeled short-chain fatty acid administered with a soluble fiber to the subject and to a healthy control are calculated. In calculating the metabolic rate a baseline first blood sample is drawn from the subject and from the healthy control after fasting. At least one stable isotope labeled short-chain fatty acid is administered intravenously to the subject and to the healthy control and a series of second blood samples are drawn at intervals from the subject and from the healthy control. A concentration of the isotope is measured in the first blood sample and in each of the series of second blood samples from the subject and from the healthy control. A compartmental analysis is applied to calculate a first metabolic rate of the at least one stable isotope labeled short-chain fatty acid from the concentrations of the isotope for the subject and for the healthy control. A soluble fiber is administered orally to the subject and to the healthy control and a second metabolic rate is calculated for the at least one stable isotope labeled short-chain fatty acid for the subject and for the healthy control as for the first metabolic rate. The metabolic rate in the subject is compared to the metabolic rate in the healthy control, where a lower metabolic rate in the presence of soluble fiber in the subject indicates a deficiency in production of short-chained fatty acids, thereby diagnosing the chronic obstructive pulmonary disease.

The present invention is further directed to a method for diagnosing autism in a subject. In this method, a metabolic rate of at least one short-chain fatty acid, at least one amino acid and at least one protein are calculated in the subject and in a healthy control. In calculating the metabolic rate after fasting, a baseline first blood sample is drawn from the subject and from the healthy control. At least one stable first isotope labeled short-chain fatty acid and at least one stable second isotope labeled amino acid are administered intravenously at intervals and at least one amino acid and a liquid nutrition formula comprising at least one stable third isotope labeled protein are administered orally at intervals. A series of second blood samples are drawn at intervals from the subject and from the healthy control. A concentration of each of the stable first isotope, the stable second isotope and the stable third isotope in the first blood sample and in each of the series of second blood samples from the subject and from the healthy control is measured. A compartmental analysis is applied to calculate the metabolic rate of the at least one short-chain fatty acid, the at least one amino acid and the at least one protein in the subject and in a healthy control. Each of the metabolic rates in the subject is compared to each of the metabolic rates in the healthy control, where a decrease in the metabolic rate of the at least one of the stable isotope labeled short-chain fatty acid, of the at least one stable second isotope labeled amino acid or of the at least one stable third isotope labeled protein or a combination thereof in the subject indicates a deficiency in production of short-chained fatty acids and quality of digestion, thereby diagnosing autism.

The present invention is directed further to a method for increasing short chain fatty acid production in an older subject. In this method, a concentration of at least one short chain fatty acid is measured in the older subject and the subject's diet is supplemented with inulin over a period of time. The concentration of the at least one short chain fatty acid is measured after supplementing to confirm an increase thereof in the older subject. The present invention is directed to a related method that further comprises repeating the measuring steps and the supplementing step.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1C show compartmental model analysis of the rate of change in blood levels of acetate, proprionate and butyrate in subjects 31A and 31B over 60 minutes after administration of stable (1,2-¹³C₂)-acetate (FIG. 1A), stable (1-¹³C)-propionate (FIG. 1B), or stable (1-¹³C)-butyrate (FIG. 1C).

FIG. 2 illustrates the procedure to examine short-chain fatty acid metabolism in Example 2.

FIG. 3 illustrates the participants flow through the procedure.

FIG. 4 shows the main side of labeled SCFA (tracer) dilution with unlabeled SCFAs (tracee) most likely occurs within colonocytes through absorption of unlabeled SCFAs produced by the intestinal microbiome. As it is assumed that labeled and unlabeled SCFAs are equally efficiently metabolized by the liver and peripheral organs, metabolism in these organs does not impact tracer-tracee ratios as long as SCFA production is negligible within these organs. SCFA production (U2) mainly represents SCFA absorption by colonocytes and is equal to metabolism (irreversible loss) of SCFA within the inaccessible pool (F02). Fractional irreversible loss is the amount of SCFAs absorbed by colonocytes or released by other organs (U2) that are metabolized within cells before being released into the systemic circulation, whereas whole-body production (WBP) is the amount of SCFAs being released into the systemic circulation after production in the inaccessible pool (U2. F₀₂. WBP. fractional irreversible loss).

FIGS. 5A-5C show the effect of 10 10 days intervention with inulin in young and older adults on the short-chain fatty acid (SCFA) production of acetate (FIG. 5A), proprionate (FIG. 5B) and butyrate (FIG. 5C) in the inaccessible pool (F₀₂).

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, “subject” or “patient” refers to a human or non-human mammal being diagnosed. As used herein, “control” or “healthy control” refers to a healthy human or non-human mammal free from any disease or disorder.

In one embodiment of the present invention, there is provided a method for determining a metabolic rate for at least one endogenous short-chain fatty acid in a subject, comprising the steps of a) drawing a baseline first blood sample from the subject; b) administering intravenously to the subject at least one stable isotope labeled short-chain fatty acid; c) drawing a series of second blood samples at intervals from the subject; d) measuring a concentration of the isotope in the first blood sample and in each of the series of second blood samples; and e) calculating a first metabolic rate of the at least one stable isotope labeled short-chain fatty acid from the concentrations of the isotope.

Further to this embodiment, the method comprises determining a deficiency in short-chain fatty acid production in the subject, comprising f) administering orally a soluble fiber to the subject; and g) repeating steps a) to e) to calculate a second metabolic rate for the at least one stable isotope labeled short-chain fatty acid; where a second metabolic rate substantially equal to the first metabolic rate indicates that short-chain fatty acid production in the subject is deficient. In this further embodiment, the soluble fiber may be inulin, oligofructose, fructooligosaccharide or a combination thereof. In this embodiment, the interval between step f) and step g) may be about 1 day to about 7 days.

In both embodiments, the subject may be fasting prior to step a). In both embodiments, the stable isotope labeled short-chain fatty acid may be ¹³C-acetate, ¹³C-propionate, ¹³C-butyrate, ¹³C-iso-butyrate, ¹³C-valerate, or ¹³C-iso-valerate. In addition, step c) may comprise drawing the series of second blood samples at intervals of about 5 minutes to about 20 minutes over a period of about 1 hour to about 5 hours. Furthermore, step e) may comprise applying a compartmental analysis to calculate the first metabolic rate.

In another embodiment of the present invention, there is provided a method for diagnosing a pulmonary disease in a subject, comprising calculating a metabolic rate of at least one stable-isotope labeled short-chain fatty acid administered with a soluble fiber to the subject and to a healthy control; and comparing the metabolic rate in the subject to the metabolic rate in the healthy control, where a lower metabolic rate in the presence of soluble fiber in the subject indicates a deficiency in production of short-chained fatty acids, thereby diagnosing the pulmonary disease.

In this embodiment, calculating the metabolic rate of the at least one short-chain fatty acid in the subject and in the healthy control comprises a) drawing a baseline first blood sample from the subject and from the healthy control; b) administering intravenously to the subject and to the healthy control at least one stable isotope labeled short-chain fatty acid; c) drawing a series of second blood samples at intervals from the subject and from the healthy control; d) measuring a concentration of the isotope in the first blood sample and in each of the series of second blood samples from the subject and from the healthy control; e) calculating a first metabolic rate of the at least one stable isotope labeled short-chain fatty acid from the concentrations of the isotope for the subject and for the healthy control; f) administering orally a soluble fiber to the subject and to the healthy control; and g) repeating steps b) to e) to calculate a second metabolic rate for the at least one stable isotope labeled short-chain fatty acid for the subject and for the healthy control. The subject and the healthy control may be fasting prior to step a). In addition, step c) may comprise drawing the series of second blood samples at intervals of about 5 minutes to about 20 minutes over a period of about 1 hour to about 5 hours. Furthermore, step e) may comprise applying a compartmental analysis to calculate the first metabolic rate. Further still an interval between step f) and step g) may be about 1 day to about 7 days.

In this embodiment, the stable isotope labeled short-chain fatty acid may be ¹³C-acetate, ¹³C-propionate, ¹³C-butyrate, ¹³C-iso-butyrate, ¹³C-valerate, or ¹³C-iso-valerate. Representative soluble fibers include but are not limited to inulin, oligofructose or fructooligosaccharide or a combination thereof. Furthermore the respiratory disorder may be chronic obstructive pulmonary disease.

In a related embodiment there is provided a method for diagnosing chronic obstructive pulmonary disease in a subject, comprising a) calculating a metabolic rate of at least one stable-isotope labeled short-chain fatty acid administered with a soluble fiber to the subject and to a healthy control, comprising i) drawing, after fasting, a baseline first blood sample from the subject and from the healthy control; ii) administering intravenously to the subject and to the healthy control at least one stable isotope labeled short-chain fatty acid; iii) drawing a series of second blood samples at intervals from the subject and from the healthy control; iv) measuring a concentration of the isotope in the first blood sample and in each of the series of second blood samples from the subject and from the healthy control; v) applying a compartmental analysis to calculate a first metabolic rate of the at least one stable isotope labeled short-chain fatty acid from the concentrations of the isotope for the subject and for the healthy control; vi) administering orally a soluble fiber to the subject and to the healthy control; and vii) repeating steps ii) to v) to calculate a second metabolic rate for the at least one stable isotope labeled short-chain fatty acid for the subject and for the healthy control; and b) comparing the metabolic rate in the subject to the metabolic rate in the healthy control, wherein a lower metabolic rate in the presence of soluble fiber in the subject indicates a deficiency in production of short-chained fatty acids, thereby diagnosing the chronic obstructive pulmonary disease.

In one aspect of this embodiment, step iii) may comprise drawing the series of second blood samples at intervals of about 5 minutes to about 20 minutes over a period of about 1 hour to about 5 hours. In another aspect an interval between step vi) and step vii) may be about 1 day to about 7 days.

In this embodiment and aspects thereof the stable isotope labeled short-chain fatty acid may be ¹³C-acetate, ¹³C-propionate, ¹³C-butyrate, ¹³C-iso-butyrate, ¹³C-valerate, or ¹³C-iso-valerate. The soluble fiber may be inulin, oligofructose or fructooligosaccharide or a combination thereof.

In yet another embodiment of the present invention, there is provided a method for diagnosing the presence of a neurological disorder in a subject, comprising calculating a metabolic rate of at least one short-chain fatty acid, at least one amino acid and at least one protein in the subject and in a healthy control; and comparing each of the metabolic rates in the subject to each of the metabolic rates in the healthy control; wherein a decrease in the metabolic rate of the at least one of the stable isotope labeled short-chain fatty acid, of the at least one stable second isotope labeled amino acid or of the at least one stable third isotope labeled protein or a combination thereof in the subject indicates a deficiency in production of short-chained fatty acids and quality of digestion, thereby diagnosing the neurological disorder.

In this embodiment, calculating the metabolic rate for the at least one short-chain fatty acid, the at least one amino acid and the at least one protein in the subject and in the healthy control comprises a) drawing a baseline first blood sample from the subject and from the healthy control; b) administering at intervals to the subject and to the healthy control at least one stable first isotope labeled short-chain fatty acid intravenously; at least one amino acid orally; at least one stable second isotope labeled amino acid intravenously; and a liquid nutrition formula comprising at least one stable third isotope labeled protein orally; c) drawing a series of second blood samples at intervals from the subject and from the healthy control; d) measuring a concentration of each of the stable first isotope, the stable second isotope and the stable third isotope in the first blood sample and in each of the series of second blood samples from the subject and from the healthy control; and e) calculating the metabolic rate of the at least one short-chain fatty acid, the at least one amino acid and the at least one protein in the subject and in a healthy control. The subject and the healthy control may be fasting prior to step a). In addition, in step b) administering the at least one stable first isotope is by pulse and the at least one stable second isotope is by primed continuous infusion. Furthermore, in step b) administering the liquid nutrition formula may be performed periodically in intervals between about every 10 minutes and about every 30 minutes for about 2 hours to about 4 hours. Further still, step c) may comprise drawing the series of second blood samples at intervals of about 5 minutes to about 20 minutes over a period of about 1 hour to about 5 hours. Further still, step e) may comprise applying a compartmental analysis to calculate the first metabolic rate.

Also in this embodiment, the stable isotope labeled short-chain fatty acid may be ¹³C-acetate, ¹³C-propionate, ¹³C-butyrate, ¹³C-iso-butyrate, ¹³C-valerate, or ¹³C-iso-valerate. In addition, the amino acid may be L-allo-a isoleucine, phenylalanine, tyrosine, leucine, tryptophan, or valine or a combination thereof. Furthermore, the stable second isotope labeled amino acid may be a ¹⁵N-L-allo-¹⁵N-isoleucine, a ¹⁵N-phenylalanine, a ¹⁵N-tyrosine, a ¹⁵N-leucine, a ¹⁵N-tryptophan, or a ¹⁵N-valine. Further still, the at least one stable third isotope labeled protein may be a ¹⁵N-labeled protein. Further still the neurological disorder is autism.

In a related embodiment, there is provided a method for diagnosing autism in a subject, comprising a) calculating a metabolic rate of at least one short-chain fatty acid, at least one amino acid and at least one protein in the subject and in a healthy control; i) drawing, after fasting, a baseline first blood sample from the subject and from the healthy control; ii) administering at intervals to the subject and to the healthy control at least one stable first isotope labeled short-chain fatty acid intravenously; at least one amino acid orally; at least one stable second isotope labeled amino acid intravenously; and a liquid nutrition formula comprising at least one stable third isotope labeled protein orally; iii) drawing a series of second blood samples at intervals from the subject and from the healthy control; iv) measuring a concentration of each of the stable first isotope, the stable second isotope and the stable third isotope in the first blood sample and in each of the series of second blood samples from the subject and from the healthy control; and v) applying a compartmental analysis to calculate the metabolic rate of the at least one short-chain fatty acid, the at least one amino acid and the at least one protein in the subject and in a healthy control; and b) comparing each of the metabolic rates in the subject to each of the metabolic rates in the healthy control; wherein a decrease in the metabolic rate of the at least one of the stable isotope labeled short-chain fatty acid, of the at least one stable second isotope labeled amino acid or of the at least one stable third isotope labeled protein or a combination thereof in the subject indicates a deficiency in production of short-chained fatty acids and quality of digestion, thereby diagnosing autism.

In one aspect of this embodiment, in step ii) administering the at least one stable first isotope may be by pulse and the at least one stable second isotope may be by primed continuous infusion. In another aspect, in step ii) administering the liquid nutrition formula may be performed periodically in intervals between about every 10 minutes and about every 30 minutes for about 2 hours to about 4 hours. In yet another aspect, step v) may comprise applying a compartmental analysis to calculate the metabolic rate.

In this embodiment and all aspects thereof, the stable isotope labeled short-chain fatty acid may be ¹³C-acetate, ¹³C-propionate, ¹³C-butyrate, ¹³C-iso-butyrate, ¹³C-valerate, or ¹³C-iso-valerate. Representative amino acids include but are not limited to L-allo-a isoleucine, phenylalanine, tyrosine, leucine, tryptophan, or valine or a combination thereof. In addition, the stable second isotope labeled amino acid may be a ¹⁵N-L-allo-¹⁵N-isoleucine, a ¹⁵N-phenylalanine, a ¹⁵N-tyrosine, a ¹⁵N-leucine, a ¹⁵N-tryptophan, or a ¹⁵N-valine. Furthermore the at least one stable third isotope labeled protein may be a ¹⁵N-labeled protein.

In yet another embodiment of the of the present invention, there is provided a method for increasing short chain fatty acid production in an older subject, comprising a) measuring a concentration of at least one short chain fatty acid in the older subject; b) supplementing the subject's diet with inulin over a period of time; and c) measuring the concentration of the at least one short chain fatty acid after supplementing to confirm an increase thereof in the older subject. Further to this embodiment, the method comprises repeating steps b) and c) at least once.

In both embodiments, steps a) to c) may comprise drawing a baseline first blood sample from the older subject after fasting to determine background concentrations of the short chain fatty acids; adding an amount of inulin to the subject's diet over the period of time; administering to the older subject ¹³C-labeled short chain fatty acids; drawing a series of second blood samples at intervals from the older subject; and applying a compartmental analysis on the series of second blood samples to determine concentrations of the ¹³C-labeled short chain fatty acids; wherein concentrations of the ¹³C-labeled short chain fatty acids greater than the background concentrations of the short chain fatty acids indicates an increase in the short chain fatty acid production in the older adult.

In both embodiments, the short chain fatty acids may be acetate, propionate and butyrate. In addition, the period of time may be about 7 days. Furthermore, the inulin supplements the subject's diet in increasing amounts over the period of the seven days.

The following example(s) are given for the purpose of illustrating various embodiments of the invention and are not meant to limit the present invention in any fashion.

### Example 1

### Analysis of Colonic Short Chain Fatty Acids in Pulmonary Disease

### 1. Recruitment, Screening & Study Day

30 COPD subjects and 30 healthy volunteers were recruited by calling previous participants on studies conducted in Center for Translational Research on Aging and Longevity-Texas A&M University, who have indicated willingness to come back for additional studies. Participants were also recruited by responding to distributed flyers, mass emails, and advertisements in the newspaper, as well as collaborations with local physicians and organizations in the community in the College Station/Bryan area. Informed consent was obtained before any study related procedures are performed. All subjects are extensively screened by research nurses/physician.

The following eligibility criteria is established for recruitment:

### 1. Inclusion Criteria for COPD Subjects

i. Ability to walk, sit down and stand up independently’
ii. Age 45-100 years;
iii. Ability to lie in supine or elevated position for 1.5 hours;
iv. Diagnosis of moderate to very severe chronic airflow limitation and compliant to the following criteria: FEV1<70% of reference FEV1;
v. Clinically stable condition and not suffering from a respiratory tract infection or exacerbation of their disease (defined as a combination of increased cough, sputum purulence, shortness of breath, systemic symptoms such as fever, and a decrease in FEV1>10% compared with values when clinically stable in the preceding year) at least 4 weeks prior to the first test day;
vi. Shortness of breath on exertion; and
vii. Willingness and ability to comply with the protocol.

### 2. Inclusion Criteria for Healthy Controls

i. Healthy male or female according to the investigator's or appointed staff's judgment;
i. Ability to walk, sit down and stand up independently;
i. Age 45-100 years;
i. Ability to lay in supine or elevated position for 1.5 hours;
i. No diagnosis of COPD; and
i. Willingness and ability to comply with the protocol.

### 3. Exclusion Criteria for all Subjects

i. Any condition that may interfere with the definition ‘healthy subject’ according to the investigator's judgment (healthy subjects only);
ii. Subjects 86 years and older that fail to get physician eligibility confirmation; Insulin dependent diabetes mellitus;
iii. Established diagnosis of malignancy;
iv. History of untreated metabolic diseases including hepatic or renal disorder;
v. Presence of acute illness or metabolically unstable chronic illness;
vi. Presence of fever within the last 3 days;
vii. Any other condition according to the PI or nurse that was found during the screening visit, that would interfere with the study or safety of the patient;
viii. Use of short course of oral corticosteroids within 4 weeks preceding first study day;
ix. Failure to give informed consent or Investigator's uncertainty about the willingness or ability of the subject to comply with the protocol requirements;
x. Pregnancy; and
xi. Already enrolled in another clinical trial and that clinical trial interferes with participating in this study.

Any recruit failing to meet the inclusion or exclusion criteria between enrollment and study day was excluded from the study. The study was conducted at a research facility of the Center for Translational Research on Aging and Longevity-Texas A&M University. The study involves one screening visit of approx. two hours, one study day of approx. three hours. On the screening visit, body weight and height were measured. Dual-energy X-ray absorptiometry (DXA) and Bioelectrical impedance analysis (BIA) were performed to measure body composition. Lung function was measured by FEV-1 and presence of lung obstruction.

### 2. Questionnaires

The following questionnaires was used to assess cognitive and mental well-being of the recruit.
i. Questionnaire about gut function and symptoms: The Gastrointestinal Symptom Rating Scale, The Gastrointestinal Symptom Rating Scale Irritable Bowel Syndrome Version.
ii. Questionnaires about health condition: COPD Assessment Test (CAT), Functional Assessment of Chronic Illness Therapy-Fatigue (FACIT-F).
iii. Questionnaire about activity: International Physical Activity Questionnaire (IPAQ).
iv. Neuropsychological tests such as the Trail Making Test, and Stroop Color-word test.

The vibrotactile behavioral battery that administers the tasks involves nine brief tests that require 20-30 minutes to administer. The vibrotactile battery involves the use of a small device that is designed to administer calibrated vibratory stimuli to the glabrous skin of digits 3 and 4 of the left hand. The battery connects to a laptop computer and participants use a computer mouse to give their responses.

### 3. Subject Health Parameters Tested

i. Vital signs: Temperature, heart rate, blood pressure, oxygen saturation was taken at various times throughout the study day or at the beginning and end of a study day.
ii. Breath analysis: Breath analysis for volatile organic compounds in exhaled breath relating to chronic disease was performed at the beginning and end of each study day.

Skeletal muscle function: Skeletal muscle function tests such as handgrip, Kin-Com (Table 1) and balance platform were assessed at screening visit and/or study day. Screening visit and study day may be combined at the recruit's convenience. Some study procedures such as body composition, skeletal muscle function, and questionnaires were skipped if completed within the past 3 months.

### 4. Blood Collection and Laboratory Analysis of Blood Sample

A temperature controlled warmed box was used to collect arterialized blood via catheter. The hand was kept within the box for the majority of the duration of the study day. The hand may be taken out briefly between blood draws.

Analysis of samples for isotope concentrations and metabolic pathways was done at the Center for Translational Research in Aging and Longevity-Texas A&M University.

TABLE 1
Kin-Com Protocol

| | | | | | Speed | Min Force |
|---|---|---|---|---|---|---|
| Protocol | Type | Duration | | Movement | (°/s) | (N) |
| Warm-up | Passive | 15 | reps | CON/CON | 50 | 1 |
| Maximal | Isokinetic | 5 | reps | CON/CON | 60 | 50 |
| Strength | | | | | | |
| Maximal | Isokinetic | 5 | re[s | CON/CON | 180 | 50 |
| Strength | | | | | | |
| Endurance | Isokinetic | 40-60 | reps* | CON/CON | 90 | 50 |

*Test to be stopped when the subject feels he/she is unable perform another repetition

### 5. Stable Isotope Infusion Protocol

Subjects were instructed to arrive in the fasted state on the study day. On the study day one catheter was inserted in the peripheral vein of an arm for blood sampling (about 16 samples in total, up to 100 ml per visit) and questionnaires related to cognition, health status, well-being, and gastrointestinal symptoms described above were completed by the subject. After a baseline blood sample is taken, a stable isotope pulse (bolus) was provided through the same line. The use of the pulse protocol enables a fast measurement without the need of careful priming or attaining steady state in the isotope enrichments. Subjects received stable isotopes solution, containing acetate (1,2-¹³C₂), propionate (1-¹³C) and butyrate (1-¹³C) using this method.

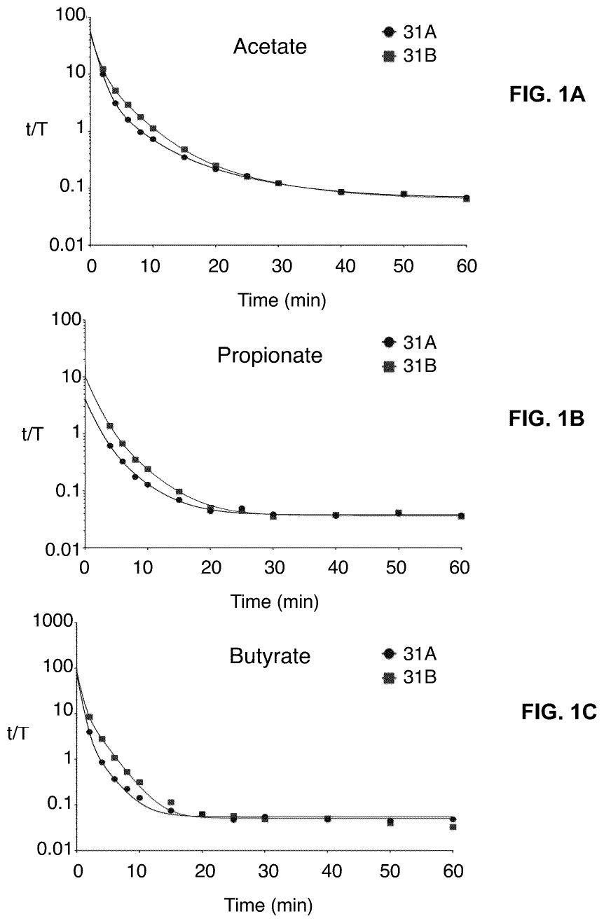

The baseline blood sample was used to establish background enrichment of stable isotopes. Samples obtained after the pulse were used to assess metabolism of short-chain fatty acid tracers. Isotope enrichments and the concentration of various metabolites were analyzed by GC-MS/MS and the colonic production of the SCFA were calculated using compartmental modelling analysis by programming and optimizing algorithms (R-language). FIG. 1A-1C shows that within 60 min, enough data points could be collected to enable the calculation of the production of acetate, propionate and butyrate.

### 6. Colonic SCFA Metabolism Analysis in Subjects

Between the first and second study day, each subject received in a randomized double blind design either Inulin (soluble fiber) and maltodextrin or maltodextrin alone (placebo) for 7 days using the regimen shown below. The pulse protocol described above is repeated each day.

To reduce side effects like bloating and diarrhea, the following escalating doses are administered twice daily:

$$\begin{matrix}Soluble Fiber group : & Day 1 : 2.5 g inulin ( IN ) + 1 g maltodextrin ( MD ) \\ & Day 2 : 5 g IN + 1 g MD \\ & Day 3 : 10 g IN + 1 g MD \\ & Day 4 - 7 : 15 g IN + 1 g MD \\ Placebo group : & Day 1 : 3.5 g MD \\ & Day 2 : 6 g MD \\ & Day 3 : 11 g MD \\ & Day 4 - 7 : 16 g MD\end{matrix}$$

The powders are weighed and packaged in individual doses by trained research staff under clean conditions and given to subjects on the first study day. Subjects were instructed to dissolve each portion of powder in 8-12 oz water and consume it at home.

### Example 2

### Analysis of Colonic Short Chain Fatty Acids, Amino Acid Metabolism and Protein Digestion in Autism

### 1. Recruitment, Screening & Study Day

30 ASD subjects and 30 healthy controls were recruited by calling previous participants on studies conducted in Center for Translational Research on Aging and Longevity-Texas A&M University, who have indicated willingness to come back for additional studies. Participants were also recruited by responding to distributed flyers, mass emails, and advertisements in the newspaper, as well as collaborations with local physicians and organizations in the community in the College Station/Bryan area. Informed consent was obtained before any study related procedures are performed. All subjects were extensively screened by research nurses/physician.

The following eligibility criteria is established for recruitment:

1. Inclusion Criteria Subjects with ASD
i. Healthy high functioning person diagnosed with autism spectrum disorder.
ii. Age 18 years to 85 years.
iii. Ability to walk, sit down and stand up independently.
iv. Ability to lie in supine or elevated position for 4 hours.
v. Willingness and ability to comply with the protocol.

### 2. Inclusion Criteria Healthy Controls

i. Healthy male or female according to the investigator's or appointed staff's judgment.
ii. Age 18 to 85 years.
iii. Ability to walk, sit down and stand up independently.
iv. Ability to lie in supine or elevated position for 4 hours.
v. Willingness and ability to comply with the protocol.

### 3. Exclusion Criteria all Subjects

i. Any condition that may interfere with the definition ‘healthy subject’ according to the investigator's judgment (for healthy group).
ii. Unwilling to comply with any other rules set forth in the Informed Consent Form Established diagnosis of Insulin Dependent Diabetes Mellitus.
iii. History of untreated metabolic diseases including hepatic or renal disorder.
iv. Presence of acute illness or metabolically unstable chronic illness.
v. Presence of fever within the last 3 days.
vi. Preplanned surgery of procedures that would interfere with the conduct of the study.
vii. Any other condition according to the PI or study physician that would interfere with proper conduct of the study/safety of the patient.
viii. Current alcohol or drug abuse.
vix. Use of protein or amino acid containing nutritional supplements within 5 days prior to the study days.
x. Use of long-term oral corticosteroids or short course of oral corticosteroids 4 weeks preceding first test day.
xi. Pregnancy.
xii. Already enrolled in another clinical trial and that clinical trial interferes with participating in this study.
xii. Montreal Cognitive Assessment (MoCA) score of <20.

Any recruit failing to meet the inclusion or exclusion criteria between enrollment and study day was excluded from the study. The study was conducted at a research facility of the Center for Translational Research on Aging and Longevity-Texas A&M University. The study involves one screening visit of approx. two hours, one study day of approx. three hours. On the screening visit, body weight and height were measured. Dual-energy X-ray absorptiometry (DXA) and Bioelectrical impedance analysis (BIA) were performed to measure body composition.

### 4. Questionnaires

The following questionnaires was used to assess cognitive and mental well-being of the recruit
i. Questionnaire about gut function and symptoms: The Gastrointestinal Symptom Rating Scale, The Gastrointestinal Symptom Rating Scale Irritable Bowel Syndrome Version.
ii. Questionnaires about sleep quality and daytime sleepiness; PSQI, Epworth Sleepiness Scale and FOSQ-10.

### 5. Cognitive Assessments:

Subjects were given tests to assess relevant cognitive functions such as cognition flexibility, attention, sensory processing, learning and memory.
i) Symbol Digit Modalities Test (DSMT) is a cognitive task developed to measure visuo motor coordination, motor persistence, sustained attention and response speed (Smith & Jones, 1982). SDMT measures attention, perceptual speed, motor speed, visual scanning and memory. Subjects are required to associate symbols with numbers and quickly generate the number when shown the symbol. Rapid information processing is required in order to substitute the symbols accurately and quickly.
ii) Digit Span (DS) tests (forward and backward) are part of the Wechsler Intelligence Scale and usually administered verbally. This test is designed to measure aspects of working memory and can be used to evaluate a variety of impairments. In this test, series of numbers of increasing length are read aloud to the subject at a rate of one digit per second. The examinee has to repeat the numbers back to them. The Digit Span test can also be administered backward. In this condition the examiner reads a list of numbers and the examinee must relay the list back in reverse order.
iii) The Montreal Cognitive Assessment (MoCA) is a cognitive screening tool designed to assist clinicians in detecting cognitive impairment. It assesses different cognitive domains: attention and concentration, executive functions, memory, language, visuoconstructional skills, conceptual thinking, calculations, and orientation (Nasreddine, Phillips et al. 2005). The English version of MoCA (available at mocatest.org) is a one-page 30-point screening test administered in 10 min to identify elderly people with mild cognitive impairment. The total possible score is 30; a score of 26 or above is considered normal; and a score below 26 with no functional impairment indicates mild cognitive impairment.
iv) The vibrotactile behavioral battery that administers the tasks involves nine brief tests that require 20-30 minutes to administer. The vibrotactile battery involves the use of a small device that is designed to administer calibrated vibratory stimuli to the glabrous skin of digits 3 and 4 of the left hand. The battery connects to a laptop computer and participants use a computer mouse to give their responses.

### 4. Subject Health Parameters Tested

i) Vital signs: Temperature, heart rate, blood pressure, oxygen saturation was taken at various times throughout the study day or at the beginning and end of a study day.
ii) Breath analysis: Breath analysis for volatile organic compounds in exhaled breath relating to chronic disease was performed at the beginning and end of each study day.
iii) Strength analysis: Handgrip strength and fatigue were assessed by handgrip dynamometry (Vernier).

Screening visit and study day are combined at the subject's convenience. Some study procedures (body composition, skeletal muscle function, strength analysis questionnaires) are skipped if completed within the past 3 months.

### 5. Blood Collection and Laboratory Analysis of Blood Sample.

A temperature controlled warmed box was used to collect arterialized blood via catheter. The hand was kept within the box for the majority of the duration of the study day. The hand may be taken out briefly between blood draws.

Analysis of samples for isotope concentrations and metabolic pathways was done at the Center for Translational Research in Aging and Longevity-Texas A&M University.

### 6. Assessment of SCFA Levels, Amino Acid Absorption and Protein Digestion

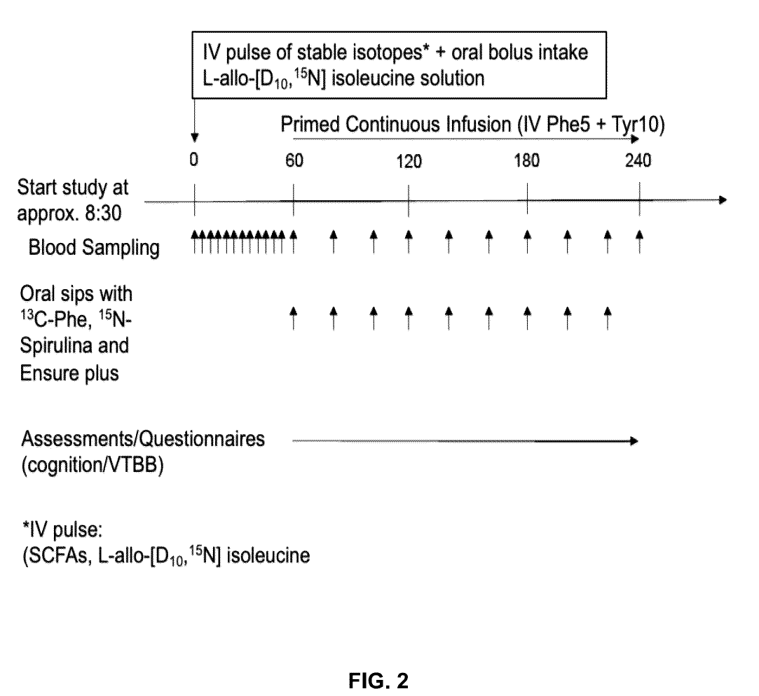

Test subjects and healthy controls were instructed to arrive in the fasted state on the study day. On the study day body weight was measured and 2 catheters (line1 and line 2) are inserted in a peripheral vein of an arm—one in each arm. One line was used for blood sampling and the other line was used for continuous infusion of stable isotopes (amino acids). After a baseline blood sample is taken, an intravenous pulse of short-chain fatty acid tracers is administered through line 1 as described for Example 1. At the same time an oral bolus of L-allo-isoleucine was administered. After 1 hour, a primed continuous infusion (PCI) of stable isotopic ¹³C-L-allo-isoleucine is initiated through line 2. The recruits were given an oral liquid nutrition formula containing stable isotope labeled (¹⁵N-labeled) spirulina proteins were administered according to a sip feeding protocol (every 20 min for 3 hours) (FIG. 2).

The baseline blood sample was used to establish background enrichment of stable isotopes. Blood samples obtained were used to quantitate short-chain fatty acid content from the abundance of stable isotope labeled SCFA (tracer). Absorption of L-allo-isoleucine was similarly detected in the same blood sample based on abundance of stable isotope labeled ¹³C-L-allo-isoleucine. Ability of the subject to digest proteins is determined by assessing the same blood sample for presence of ¹⁵N-labeled amino acids (formed by digestion of ¹⁵N-labeled spirulina proteins).

Isotope enrichments and the concentration of various metabolites were analyzed by GC-MS/MS. Compartmental modelling analysis as described in Example 1 is used to determine content of the tracers in the blood sample which gives an assessment of SCFA content, amino acid content and protein digestibility in the subject using a single protocol. This approach is beneficial since it avoids multiple hospital visits and provides a single stage diagnostic analysis of a potential autism subject's metabolic profile. The above described method may be use for detecting content of any short-chain fatty acid, digestion of any protein of interest or absorption of any amino acid and can be implemented in a single clinical protocol.

### Example 3

### Inulin Supplementation Increases Short-Chain Fatty Acid Kinetics and Concentrations in Young and Older Adults: Methods

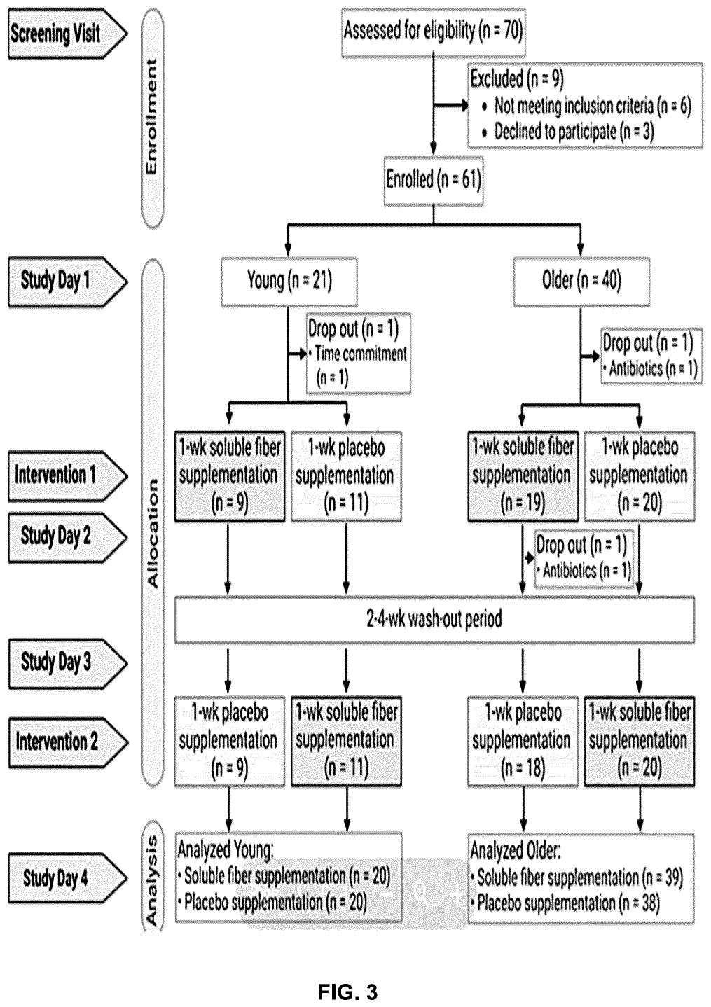

Seventy-one adults were assessed for eligibility of which sixty-two were enrolled 62 (FIG. 3). Participants were recruited for participation if they were between the ages of 18 and 30 (young group) or 45 and 100 (older group), were able to walk, sit down and stand up independently, and reported that they were willing to comply with the protocol and lie in supine or elevated position for 1.5 h. Participants were excluded because of vein access issues or a condition that impacts intestinal metabolism or microbiome composition (malignancy, insulin-dependent diabetes, (possible) pregnancy, metabolic, renal or hepatic disease, recent surgery, fever last 3 d), intake of additional pre/probiotics (last week), oral corticosteroids (last 4 wk), antibiotics (last 3 mo).

All individuals provided a written informed consent before participation in the study. We assessed medication intake and comorbidities by interview and medical records review. Enrollment of participants was ongoing for 19 months. Eligible participants returned for 4 test days (˜2 h each) separated by 2 1-wk intervention periods of inulin and maltodextrin intake in randomly assigned order between test days 1 and 2 or days 3 and 4 62 (FIG. 3). Study visits were completed over a period of 21 months.

The 1-wk nutritional interventions were performed in a randomized, double-blind, placebo-controlled, crossover design and consisted of 1) inulin (fructooligosaccharide) (mean DP: 6-8; Piping Rock Health Products, LLC), and 2) maltodextrin (BulkSupplements.com, Hard Eight Nutrition LLC). Food and Drug Administration (FDA) defines inulin as an isolated, nondigestible carbohydrate with beneficial physiological effects to human health (1). Maltodextrin was selected as placebo due to the similar appearance and comparable taste as inulin. Randomization was by stratified permuted block randomization using randomizer.org.

Both powders were orally administered by participants, one dose in the morning and one in the evening, and doses increased throughout the intervention: days 1-2:5 g (10 g/d); day 3:7.5 g (15 g/d); day 4:10 g (20 g/d); day 5:12.5 g (25 g/d); days 6-7:15 g (30 g/d). The final daily powder dose was based on the FDA recommendations to ingest ≥28 g fiber/d (2). The participants consumed 15 g in the evening of day 7 and was followed the next morning with the SCFA isotope studies on day 8. An independent researcher was responsible to blind the powder labels. Powders, identical in appearance, were provided in bottles labeled with “day nr (1-7) morning/evening” and could be diluted in drinks or added to food. Participants recorded time of intake.

Participants were instructed to maintain their dietary habits and to report their dietary intake (habitual dietary caloric, fat, protein, carbohydrate, and fiber intake) in the week before and during each intervention using 3-day food records (3). All food/drink consumed for 3 days, including 2 weekdays and 1 weekend day, was to be included in the record.

BMI was calculated and total fat mass, fat-free mass, lean body mass, and appendicular skeletal muscle mass (sum of muscle mass in legs and arms) were measured by dual-energy x-ray absorptiometry (DEXA) at the screening visit (4, 3).

Participants were studied after an overnight fast and arrived at the clinical research unit about 08:00 A.M. The gastrointestinal symptom rating scale (GSRS), stool consistency (Bristol Stool Scale) and Profile of Mood States were assessed.

For SCFA kinetics measurement, one IV catheter was inserted into a superficial vein of the lower arm and the hand was placed in a thermostatically controlled hot box (internal temperature: 50° C.) for arterialized-venous blood sampling. Baseline blood was sampled to determine acetate, propionate, and butyrate background enrichments and SCFA and BCFA concentrations. At t=0 min, an 8.48 [95% confidence interval (CI): 8.44, 8.51] mL pulse containing 62.3 mmol/L acetate [¹³C₂], 6.8 mmol/L propionate [¹³C₃], and 5.5 mmol/L butyrate [¹³C₄] (microbiological was infused through the same catheter and pyrogen tested, Cambridge Isotope Laboratories), made iso-osmolar by NaCl. Arterialized-venous blood was subsequently sampled 4 times over a 30 min period at t=4, 8, 15, and 30 min (4).

Participants collected fecal samples at home <24 h before each test day, stored in a cooler box at 4° C. until dropped off at our facility. If no fecal sample could be collected before a test day, adults collected a fecal sample before supplementation was started or postintervention continued supplementation until fecal sample collection.

Arterialized-venous blood was collected in EDTA tubes, immediately stored them on ice, obtained plasma through centrifugation (4° C., 8000 g for 5 min), and stored plasma at 80° C. until analysis. Stool samples were manually homogenized and aliquots stored at 80° C. within 24 h of collection. 1) Enrichments of the used SCFA tracers and 2) fecal (μmol/g dry weight) and 3) plasma SCFA (acetate, propionate, butyrate, valerate) and BCFA (isobutyrate, isovalerate, 3-methylbutyrate) concentrations were measured batchwise by GC-MS of their pentafluorophenyl esters.

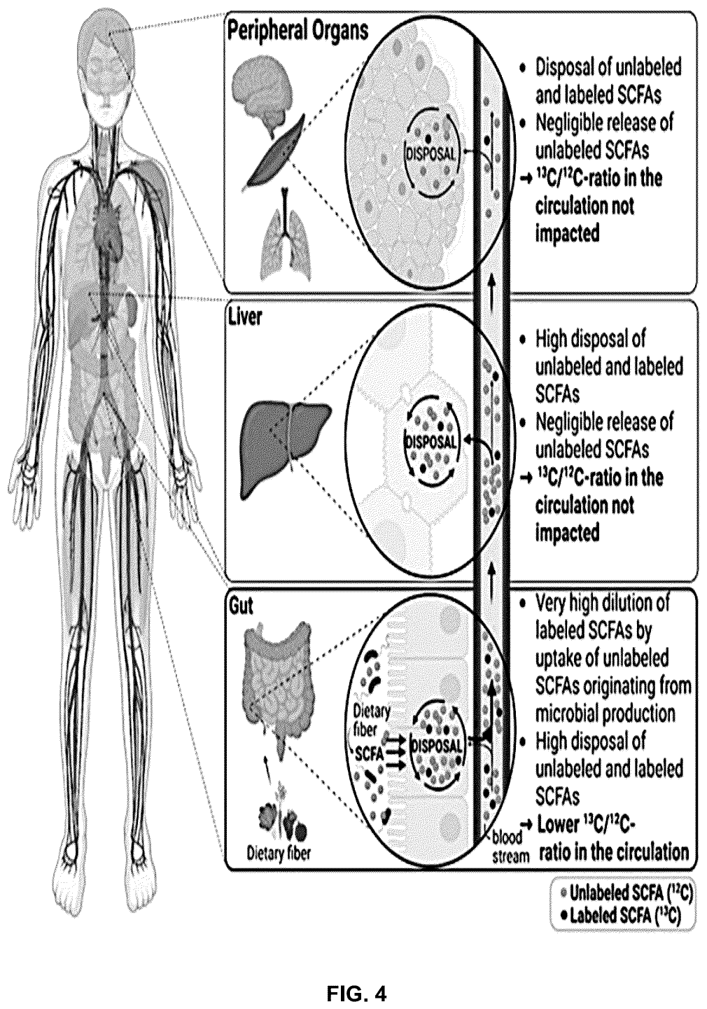

SCFA compartmental parameters were calculated with compartmental modeling (4): whole-body production (WBP) (4), SCFA pool sizes, production within these pools, and fluxes between the pools. It was assumed that SCFA production and disposal only occur in the inaccessible pool (Pool 2), probably mainly representing colonocytes absorbing SCFAs after microbial production (4) (FIG. 4). In compartmental modeling, the fluxes between the pools are equal (F₂₁=F₁₂) and the production in Pool Q₂ is equal to the disposal in Q₂.

### Statistics

All results are expressed as mean (SD) or (95% CI). The study population characteristics are shown in Table 2. Preintervention group differences were assessed by generalized linear model and it was found that the gamma distribution and the log link needed to be used and covariates biological sex and fiber intake were included. To study the intervention effect on compartmental parameters, a generalized linear mixed model with fixed effects was used: study day (before or after treatment), intervention (placebo or Inulin), group (young or older adults), fiber intake and random effects: subject code. The gamma family with Log link was used. Missing data were not imputed as these tests can handle missing values. Regression analysis of log preintervention WBP and U2 with log plasma and fecal concentrations was performed. The significance level was set to α=0.05 and statistical significance as P<α. All statistical analyses were performed using JASP (0.19.3) (5) that is based on R.

TABLE 2
Study population characteristics

| | Young adults | | Older adults | | |
|---|---|---|---|---|---|
| | (n = 21) | | (n = 40) | | P value |
| General Characteristics | | | | | |
| Age (y) | 23.21 | (2.77) | 7.20 | (7.41) | <0.001 |
| Biological sex (n, male/female) | 12/10 | | 19/21 | | 1.000 |
| BMI, kg/m² | 24.21 | (3.73) | 27.71 | (4.25) | 0.002 |
| Fat mass index, kg/m² | 5.72 | (2.64) | 9.07 | (3.14) | <0.001 |
| Visceral adipose tissue mass | 268.7 | (142.3) | 782.5 | (339.7) | <0.001 |
| (g) | | | | | |
| Fat-free mass index, kg/m² | 17.06 | (2.63) | 17.10 | (2.65) | 0.951 |
| Appendicular skeletal muscle | 7.69 | (1.65) | 7.10 | (1.28) | 0.162 |
| index, kg/m² | | | | | |
| Charlson comorbidity index | 0 | (0) | 0.25 | (0.543) | <0.001 |
| Physical Activity Scale for the | 158.2 | (35.36) | 189.1 | (50.76 | 0.001 |
| Elderly (score) | | | | | |
| High-sensitivity C-reactive | 0.45 | (0.40) | 1.41 | (1.29) | <0.001 |
| protein, mg/L | | | | | |
| Transcutaneous oxygen | 98.29 | (0.72) | 97.23 | (1/37) | 0.003 |
| saturation (%) | | | | | |
| Habitual dietary intake | | | | | |
| Calories, kcal/d | 1984 | (366.6) | 2055 | (533.9) | 0.541 |
| Carbohydrate (g/d) | 238.4 | (45.34) | 233.9 | (79.32) | 0.782 |
| Protein (g/d) | 78.21 | (26.14) | 77.43 | (27.15) | 0.914 |
| Fat (g/d) | 77.03 | (25.85) | 82.03 | (28.05) | 0.407 |
| Fiber (g/d) | 15.46 | (8.06) | 20.75 | (10.81) | 0.036 |

Values are means (SD) except for sex
Statistics by Welch t-test or binomial test (biological sex, only)

### Example 4

### Inulin Supplementation Increases Short-Chain Fatty Acid Kinetics and Concentrations in Young and Older Adults: Results

### Population Characteristics

21 young (age range: 20-29 y) and 40 older (age range: 59-87 y) adults were studied. The 4 dropouts (6.5%) were due to factors unrelated to the supplement intake: 2 adults were no longer meeting the inclusion criteria after test day 1 (antibiotics treatment), 1 was an early discontinuation during test day 1 due to difficulty with the IV catheter (no metabolism data collected), 1 voluntarily withdrew after test day 1 due to time commitment issues (FIG. 1).

Both groups (Table 2) had similar sex distributions but older adults had a higher BMI due to more fat mass as reflected by a higher fat mass index and visceral adipose tissue mass. Fat-free mass index and appendicular skeletal muscle index, a measurement for the presence of sarcopenia, as well as habitual caloric, carbohydrate, protein, and fat intake were comparable between the groups with a higher fiber intake in the older adults. Older adults had more (severe) comorbidities as indicated by Charlson comorbidity scores but none of the adults suffered from any ongoing severe comorbidities (Charlson comorbidity index>2). Overall, young adults did not report any comorbidities besides psychological issues (n=3, 14%). The most common comorbidities in the older adults were osteoarthritis (n=25, 63%), hypertension (n=18, 45%), dyslipidemia (n=18, 45%), hypothyroidism (n=15, 38%) and psychological issues (n=7, 18%). Older adults reported a higher physical activity level and had lower transcutaneous oxygen saturation. Although older adults had higher hsCRP concentrations than young adults, none of the groups was characterized by low-grade inflammation.

### Compliance and Side Effects

Preintervention Total Mood Disturbance score and self-reported gastrointestinal symptoms (total score, and subscores of diarrhea, indigestion, constipation, abdominal pain, as well as reflux), as well as self-rated stool consistency did not differ between young and older adults. Inulin and placebo supplementations did not differently affect Total Mood Disturbance. However, young and older adults suffered from more gastrointestinal symptoms after inulin than placebo supplementation (GSRS total score), in particular due to an increased feeling of indigestion, as well as a higher diarrhea score. Bristol Stool Scale after interventions did not differ between young and older adults.

Three young and 9 older adults missed 1-2 inulin doses and 7 young and 9 older adults missed 1-2 placebo doses, but were not excluded from the analysis. Two young adults missed 3-4 inulin doses due to reasons unrelated to the side effects (spontaneous travel; mouth ulcer). The number of supplement doses taken did not differ between any groups (data not shown).

Self-reported intake of calories, carbohydrates, protein, fat, and fiber during interventions was comparable between groups and interventions with the exception of a lower carbohydrate intake during inulin and placebo supplementation.

### SCFA Compartmental Parameters

Older adults (Table 3) had lower preintervention acetate [954 (1445, 463); P=<0.001], and butyrate [14.6 (25.7, 3.44); P=0.016] production rates (μmol/min) in the inaccessible pool (U2. F₀₂), potentially representing the pool in which SCFAs produced by the intestinal microbiome drain into. Furthermore, older adults had smaller acetate [6642 (10037, 3247); P<0.001] and butyrate [63.7 (112.2, 15.3); P=0.015] pool sizes (μmol) of the inaccessible pool (Q2). No differences were observed in propionate production in the inaccessible pool and pool size of Q2. Clearance of acetate [0.576 (0.343, 1.495); P=0.008] and butyrate [0.674 (0.078, 1.27); P=0.034] from the systemic circulation was lower in older adults.

TABLE 3
Baseline short-chain fatty acid kinetics in young and older adults

| | | | Older adults − | |
|---|---|---|---|---|
| | Young adults | Older adults | young adults | P value |
| Acetate | | | | |
| Whole-body production, | 169.6 | 161.1 | −8.4 | 0.66 |
| μmol/min | 140.6, 204.3 | 140.4, 184.8 | −45.8, 28.9 | |
| Size of accessible pool | 291.9 | 338.0 | 47 | 0.286 |
| (Q₁), μmol | 233.4, 365.2 | 285.1, 402.8 | −37.7, 131.6 | |
| Flux between pools | 177.4 | 197.9 | 20.5 | 0.416 |
| F₂₁ = F₁₂), μmol/min | 143, 220.1 | 167.6, 233.7 | −28.2, 69.2 | |
| Size of inaccessible | 12662 | 6019 | −6642 | <0.001 |
| pool, (Q₂), μmol | 9739, 16461 | 4938, 7337 | −10037,3247 | |
| Production in | 1888 | 934 | −954 | <0.001 |
| inaccessible pool (U₂ = | 1465, 2432 | 771, 1130 | −1445, −463 | |
| F₀₂), μmol/min | | | | |
| Ratio Q₁/Q₂ | 0.03 | 0.059 | 0.028 | <0.001 |
| | 0.023, 0.04 | 0.048, 0.072 | 0.014, 0.042 | |
| Plasma clearance | 1.869 | 2.598 | 0.729 | 0.055 |
| | 1.419, 2.463 | 2.122, 3.182 | 0.016, 1.42 | |
| Propionate | | | | |
| Whole-body production, | 3.1 | 4.47 | 1.37 | 0.047 |
| μmol/min | 2.31, 4.16 | 3.58, 5.59 | 0.07, 2.67 | |
| Size of accessible pool | 4.2 | 6.68 | 2.48 | 0.042 |
| (Q₁), μmol | 2.92, 6.02 | 5.08, 8.78 | 0.19, 4.77 | |
| Flux between pools | 3.45 | 5.31 | 1.86 | 0.042 |
| F₂₁ = F₁₂), μmol/min | 2.47,4.83 | 4.12,6.84 | 0.15, 3.57 | |
| Size of inaccessible | 191.9 | 142.4 | −49.4 | 0.06 |
| pool, (Q₂), μmol | 151.9, 242.4 | 110.4, 169.9 | −98.9, 0.1 | |
| Production in | 43.25 | 33.17 | −10.07 | 0.081 |
| inaccessible pool (U₂ = | 34.48, 54.25 | 27.96, 39.36 | −20.08, 0.83 | |
| F₀₂), μmol/min | | | | |
| Ratio Q₁/Q₂ | 0.026 | 0.049 | 0.023 | 0.008 |
| | 9.918, 0.037 | 0.036, 0.064 | 0.007, 0.039 | |
| Plasma clearance | 2.343 | 2.919 | 0.576 | 0.229 |
| | 1.741, 3.152 | 2.333, 3.652 | −0.343, 1.495 | |
| Butyrate | | | | |
| Whole-body production, | 1.03 | 1.2 | 0.17 | 0.47 |
| μmol/min | 0.74, 1.44 | 0.93, 1.54 | −0.28, 0.61 | |
| Size of accessible pool | 1.15 | 1.4 | 0.26 | 0.351 |
| (Q₁), μmol | 0.81, 1.63 | 1.08, 1.83 | −0.27, 0.79 | |
| Flux between pools | 1.08 | 1.3 | 0.22 | 0.38 |
| F₂₁ = F₁₂), μmol/min | 0.76, 1.53 | 1, 1.7 | −0.27, 0.71 | |
| Size of inaccessible | 137.7 | 74 | −63.7 | 0.015 |
| pool, (Q₂), μmol | 98, 193.6 | 57.9, 94.5 | −112.2, −15.3 | |
| Production in | 32 | 17.42 | −14.58 | 0.016 |
| inaccessible pool | 22.86, 44,8 | 13.68, 22.19 | −25.72, −3.44 | |
| (U₂ = F₀₂), μmol/min | | | | |
| Ratio Q₁/Q₂ | 0.01 | 0.021 | 0.011 | 0.002 |
| | 0.007, 0.014 | 0.017, 0.027 | 0.005, 0.017 | |
| Plasma clearance | 0978 | 1.652 | 0.674 | 0.034 |
| | 0.662, 1.444 | 1.231, 2.218 | 0.078, 1.27 | |

Values are estimated marginal means (95% CI). Statistics were performed by generalized linear model with family: gamma and link: log. Covariates included biological sex, and fiber intake.
Abbreviations: CI, confidence interval; Fᵢⱼ, trace flux from compartment j to ᵢ; Qᵢ, trace pool size in compartment i; U₂, tracee production in compartment 2 (that is, F₀₂).

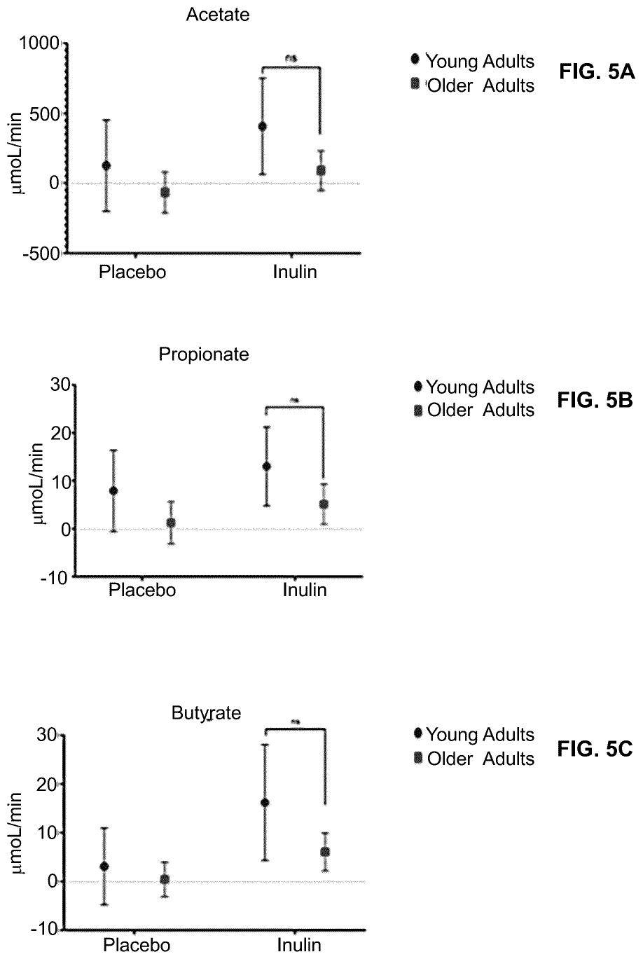

Inulin supplementation did not result in an increase in production in the inaccessible pool and the other compartmental analysis parameters of acetate (Table 4 3 and FIGS. 5A-5C).

### TABLES 4A-4C

One-week inulin and placebo intervention-induced changes in acetate (C2) kinetics in young and older adults

| | | | Young |
|---|---|---|---|
| | Young | Young | adults-placebo |
| | adults-placebo | adults-placebo | Delta post v. |
| | Pre-intervention | Post-intervention | pre-intervention |
| Whole body | 165 | 164 | −0.9 |
| production | (132, 206) | (127, 212) | (−25.1, 23.4) |
| μmol/min | | | P = 1 |
| Size of | 296 | 307 | 10.9 |
| inaccessible pool, | (232, 378) | (248, 379) | (−47.4, 69.2) |
| (Q1), μmol | | | P = 0.0998 |
| Flux between | 179 | 186 | 7.2 |
| pools | (141, 227) | (151, 229) | (−26.1, 40.6) |
| F₂₁ = F₁₂, | | | P = 0.996 |
| μmol/min | | | |
| Size of | 9877 | 10768 | 891 |
| inaccessible pool, | (7613, 12815) | (8678, 13363) | (−1409.9, 3191.9) |
| (Q2), μmol | | | P = 0.949 |
| Production in | 1497 | 1623 | 125.5 |
| inaccessible pool | (1167, 1921) | (1315, 2003) | (−200.7, 451.7) |
| (U₂ = F₀₂), | | | P = 0.95 |
| μmol/min | | | |
| Ratio Q₁/Q₂ | 0.027 | 0.029 | 0.002 |
| | (0.02, 0.037) | (0.023, 0.036) | (−0.006, 0.01) |
| | | | P = 0.995 |
| Clearance | 2.042 | 2.472 | 0.43 |
| L/min | (1.583, 2.633) | (1.919, 3.185) | (−0.101, 0.962) |
| | | | P = 0.449 |
| | | | Young |
| | Young | Young | adults-inulin |
| | adults-inulin | adults-inulin | Delta post v. |
| | Pre-intervention | Post-intervention | pre-intervention |
| Whole body | 169 | 202 | 32.4 |
| production | (134, 214) | (156, 262) | (2.7, 62.2) |
| μmol/min | | | P = 0.153 |
| Size of | 331 | 389 | 57.7 |
| inaccessible pool, | (25, 433) | (308, 490) | (−10.9, 126.3) |
| (Q1), μmol | | | P = 0.407 |
| Flux between | 197 | 235 | 37.7 |
| pools | (153, 255) | (188, 294) | (−1.4, 76.7) |
| F₂₁ = F₁₂, | | | P = 0.26 |
| μmol/min | | | |
| Size of | 9335 | 12181 | 2825.5 |
| inaccessible pool, | (7107, 12314) | (9667, 15349) | (423.9, 5227) |
| (Q2), μmol | | | P = 0.101 |
| Production in | 1438 | 1845 | 407.2 |
| inaccessible pool | (1109, 1864) | (1478, 2304) | (63, 751.4) |
| (U₂ = F₀₂), | | | P = 0.098 |
| μmol/min | | | |
| Ratio Q₁/Q₂ | 0.034 | 0.035 | 0.001 |
| | (0.024, 0.048) | (0.027, 0.046) | (−0.009, 0.011) |
| | | | P = 1 |
| Clearance | 1.896 | 2.118 | 0.221 |
| L/min | (1.445, 2.489) | (1.617, 2.773) | (−0.245, 0.688) |
| | | | P = 0.886 |

TABLE 4B

| | Older | Older | Older |
|---|---|---|---|
| | adults-placebo | adults-placebo | adults-placebo |
| | Pre- | Post- | Delta post v. |
| | intervention | intervention | pre-intervention |
| Whole body | 151 | 144 | −7.4 |
| production | (129, 178) | (120, 172) | (−26.5, 11.7.25)) |
| μmol/min | | | P = 0.949 |
| Size of | 298 | 300 | 1.7 |
| inaccessible pool, | (249. 358) | (257, 349) | (−43.1, 46.4) |
| (Q1), μmol | | | P = 1 |
| Flux between | 176 | 176 | 0 |
| pools F₂₁ = F₁₂, | (147, 210) | (152, 204) | (−25.1, 25) |
| μmol/min | | | P = 1 |
| Size of | 6123 | 5686 | −437.1 |
| inaccessible pool, | (5053, 7420) | (4855, 6660) | (−1442.6, 568.4) |
| (Q2), μmol | | | P = 0.918 |
| Production in | 950 | 884 | −65.4 |
| inaccessible pool | (790, 1142) | (758, 1032) | (−211.7, 80.9) |
| (U₂ = F₀₂), | | | P = 0.909 |
| μmol/min | | | |
| Ratio Q₁/Q₂ | 0.047 | 0.052 | 0.005 |
| | (0.037, 0.059) | (0.044, 0.061) | (−0.005, 0.015) |
| | | | P = 0.879 |
| Clearance | 2.509 | 2.918 | 0.41 |
| L/min | (2.053, 3.066) | (2.398, 3.552) | (−0.053, 0.972) |
| | | | P = 0.35 |
| | Older | Older | Older |
| | adults-inulin | adults-inulin | adults-inulin |
| | Pre- | Post- | Delta post v. |
| | intervention | intervention | pre-intervention |
| Whole body | 134 | 152 | 18.6 |
| production | (113, 158) | (127, 182) | (0.4, 36.8) |
| μmol/min | | | P = 0.206 |
| Size of | 271 | 309 | 37.6 |
| inaccessible pool, | (224, 328) | (262, 364) | −(3.9, 79.2) |
| (Q1), μmol | | | P = 0.327 |
| Flux between | 159 | 182 | 23 |
| pools F₂₁ = F₁₂, | (132, 191) | (155, 213) | (−0.2, 46.1) |
| μmol/min | | | P = 0.235 |
| Size of | 5615 | 6228 | 612.2 |
| inaccessible pool, | (4606, 6846) | (5290, 7332) | (−351.2, 1575.7) |
| (Q2), μmol | | | P = 0.698 |
| Production in | 879 | 969 | 90 |
| inaccessible pool | (729, 1060) | (829, 1133) | (−51.2, 231.2) |
| (U₂ = F₀₂), | | | P = 0.695 |
| μmol/min | | | |
| Ratio Q₁/Q₂ | 0.046 | 0.049 | 0.003 |
| | (0.036, 0.059) | (0.041, 0.059) | (−0.007, 0.013) |
| | | | P = 0.98 |
| Clearance | 2.366 | 2.539 | 0.173 |
| L/min | (1.92, 2.915) | (2.063, 3.124) | (−0.239, 0.584) |
| | | | P = 0.929 |

TABLE 4C

| | | | ANOVA |
|---|---|---|---|
| | Post hoc | ANOVA | Study day* |
| | Inulin | Study day | Intervention |
| | (young-older) | Intervention | Study day* group |
| | (post-pre) | group | Intervention* group |
| Whole body | 6.9 | 0.149 | 1 |
| production | (−9.7, 23.4) | 0.429 | 0.577 |
| μmol/min | P = 0.931 | 1 | 0.169 |
| Size of | 10 | 0.156 | 0.056 |
| inaccessible pool, | (−27.5, 47.6) | 0.28 | 0.79 |
| (Q1), μmol | P = 0.99 | 0.367 | 0.115 |
| Flux between | 7.4 | 0.117 | 0.03 |
| pools F₂₁ = F₁₂, | (−13.9, 28.6) | 0.28 | 0.717 |
| μmol/min | P = 0.968 | 0.268 | 0.101 |
| Size of | 1106.6 | 0.176 | <.001 |
| inaccessible pool, | (−153, 2366.2) | 0.771 | 0.252 |
| (Q2), μmol | P = 0.359 | <.001 | 0.799 |
| Production in | 158.6 | 0.175 | <.001 |
| inaccessible pool | (−22.1, 339.3) | 0.671 | 0.246 |
| (U₂ = F₀₂), | P = 0.36 | <.001 | 0.759 |
| μmol/min | | | |
| Ratio Q₁/Q₂ | −0.001 | 0.464 | 0.581 |
| | (−0.008, 0.006) | 0.338 | 0.846 |
| | P = 0.000 | 0.001 | 0.166 |
| Clearance | 0.024 | 0.167 | 0.113 |
| L/min | (−0.278, 0.327) | 0.283 | 0.167 |
| | P = 1 | 0.055 | 0.283 |

Values are estimated marginal means and estimated marginal mean differences between post- and preintervention data with 95% CI. Statistics were performed by generalized linear mixed model with fixed effects:study day (before or after treatment), intervention (placebo or inulin), group (young or older adults), fiber intake and random effects: subject code. The gamma family with Log link was used. Turkey was used to correct the P values for multiple comparisons. Abbreviations: ANOVA, analysis of variance; CI confidence interval; Fᵢⱼ tracee flux from compartment j to i; Qᵢ , tracee pool size in compartment i; U₂, tracee production in compartment 2 (that is, F₀₂).

In contrast, increased production in the inaccessible pool in young adults of propionate [0.13.0 (4.8, 21.1); P<0.01] and butyrate [0.16.2 (4.3, 28.1); P. 0.038] and older adults of butyrate [0.6.1 (2.2, 9.9); P<0.011] was found after inulin supplementation (Tables 5 and 6, FIGS. 5A-5C). However, differences in the response between young and older adults were not found.

### TABLES 5A-5C

One-week inulin and placebo intervention-induced changes in acetate (C3) kinetics in young and older adults

| | Young | Young | Young |
|---|---|---|---|
| | adults-placebo | adults-placebo | adults-placebo |
| | Pre- | Post- | Delta post v. |
| | intervention | intervention | pre-intervention |
| Whole body | 3.43 | 3.84 | 0.41 |
| production | (2.62, 4.51) | (2.96, 4.99) | (−0.35, 1.17) |
| μmol/min | | | P = 0.822 |
| Size of | 4, 6 | 5.14 | 0.3 |
| inaccessible pool, | (3, 37, 6,26) | (3.86, 6.82) | (−0.67, 1.73) |
| (Q1), μmol | | | P = 0.911 |
| Flux between | 3.81 | 4.23 | 0.43 |
| pools | (3.48, 4.17) | (3.84, 4.66) | (0.33, 0.52) |
| F₂₁ = F₁₂, | | | P = <.001 |
| μmol/min | | | |
| Size of | 187.7 | 226 | 38.3 |
| inaccessible pool, | (149.1, 236.3) | (184.99, 276.09) | (−2.28, 78.87) |
| (Q2), μmol | | | P = 0.283 |
| Production in | 43 | 50.9 | 7.91 |
| inaccessible pool | (34.53, 53.55) | (41.68, 62.18) | (−0.55, 16.37) |
| (U₂ = F₀₂), | | | P = 0.293 |
| μmol/min | | | |
| Ratio Q₁/Q₂ | 9,924 | 0.023 | 0 |
| | (0.023, 0.024) | (0.023, 0.024) | (−0.001, 0) |
| | | | P = <.001 |
| Clearance | 2,237 | 2.614 | 0.377 |
| L/min | (1.773, 2.825) | (2.1, 3.254) | (−0.223, 0.976) |
| | | | P = 0.708 |
| | Young | Young | Young |
| | adults-inulin | adults-inulin | adults-inulin |
| | Pre- | Post- | Delta post v. |
| | intervention | intervention | pre-intervention |
| Whole body | 3.78 | 4.17 | 0.4 |
| production | (2.81, 5.08) | 3.15, 5.53) | (−0.44, 1.23) |
| μmol/min | | | P = 0.886 |
| Size of | 5.38 | 5.59 | 0.21 |
| inaccessible pool, | (3.81, 7.58) | 4.06, 7.68) | (−1.15, 1.56) |
| (Q1), μmol | | | P = 0.999 |
| Flux between | 4.32 | 4.62 | 0.3 |
| pools | (3.96, 4.72) | 4.21, 5.07) | (0.2, 0.4) |
| F₂₁ = F₁₂, | | | P = <.001 |
| μmol/min | | | |
| Size of | 161.45 | 223.97 | 62.52 |
| inaccessible pool, | (123.47, | 176.79, 283.72) | (24.06, 100.98) |
| (Q2), μmol | 211.11) | | P = 0.007 |
| Production in | 38.13 | 51.12 | 13 |
| inaccessible pool | (29.58, 49.15) | 40.51, 64.52) | (4.79, 21.21) |
| (U₂ = F₀₂), | | | P = 0.01 |
| μmol/min | | | |
| Ratio Q₁/Q₂ | 0.031 | 0.025 | −0.006 |
| | (0.03, 0.032) | 0.024, 0.026) | (−0.007, −0.006) |
| | | | P = <.001 |
| Clearance | 2.344 | 2.509 | 0.165 |
| L/min | (1.772, 3.101) | (1.929, 3.263) | (−0.434, 0.764) |
| | | | P = 0.988 |

TABLE 5B

| | Older | Older | Older |
|---|---|---|---|
| | adults- | adults- | adults- |
| | placebo | placebo | placebo |
| | Pre- | Post- | Delta post v. |
| | intervention | intervention | pre-intervention |
| Whole body | 4.05 | 4.69 | 0.65 |
| production | (3.31, 4.94) | (3.88, 5.67) | (−0.05, 1.34) |
| μmol/min | | | P = 0.296 |
| Size of | 5.81 | 6.97 | 1.16 |
| inaccessible pool, | (4.62, 7.32) | (5.65, 8.6) | (−0.04, 2.35) |
| (Q1), μmol | | | P = 0.256 |
| Flux between | 4.67 | 5.53 | 0.87 |
| pools F₂₁ = F₁₂, | (4.25, 5.13) | (5.04, 6.07) | (0.74, 0.00) |
| μmol/min | | | P = <.001 |
| Size of | 138 | 142.14 | 4.15 |
| inaccessible pool, | (116.56, | (122.82, | (−16.49, 24.78) |
| (Q2), μmol | 163.37) | 164.5) | P = 0.997 |
| Production in | 32.16 | 33.43 | 1.27 |
| inaccessible pool | (27.38, 37.78) | (28.89, 38.69) | (−3.14, 5.69) |
| (U₂ = F₀₂), | | | P = 0.986 |
| μmol/min | | | |
| Ratio Q₁/Q₂ | 0.041 | 0.049 | 0.008 |
| | (0.04, 0.043) | (0.048, 0.51) | (0.008, 0.008) |
| | | | P = <.001 |
| Clearance | 2.641 | 2.984 | 0.343 |
| L/min | (2.207, 3.159 | (2.534, 3.514 | −0.17, 0.856 |
| | | | P = 0.651 |
| | Older | Older | Older |
| | adults-inulin | adults-inulin | adults-inulin |
| | Pre- | Post- | Delta post v. |
| | intervention | intervention | pre-intervention |
| Whole body | 3.6 | 4.12 | 0.52 |
| production | (2.91, 4.46) | (3.36, 5.05) | (−0.09, 1.13) |
| μmol/min | | | P = 0.385 |
| Size of | 5.26 | 5.87 | 0.61 |
| inaccessible pool, | (4.1, 6.76) | (4.67, 7.39) | (−0.41, 1.64) |
| (Q1), μmol | | | P = 0.75 |
| Flux between | 4.15 | 4.73 | 0.58 |
| pools F₂₁ = F₁₂, | (3.79, 4.54) | (4.32, 5.18) | (0.48, 0.68) |
| μmol/min | | | P = <.001 |
| Size of | 123.45 | 146.51 | 23.06 |
| inaccessible pool, | (101.68, | (123.48, | 3(.43, 42.69) |
| (Q2), μmol | 149.87 | 173.83 | P = 0.102 |
| Production in | 28.84 | 33.96 | 5.12 |
| inaccessible pool | (24, 34.66) | (28.7, 40.18) | (0.01, 0.33) |
| (U₂ = F₀₂), | | | P = 0.082 |
| μmol/min | | | |
| Ratio Q₁/Q₂ | 0.041 | 0.04 | −0.001 |
| | (0.04, 0.042) | (0.039, 0.041) | (−0.002, −0.001) |
| | | | P = <.001 |
| Clearance | 2.381 | 2.465 | 0.085 |
| L/min | (1.936, 2.927) | (2.032, 2.99) | (−0.66, 0.634) |
| | | | P = 0.998 |

TABLE 5C

| | | | ANOVA |
|---|---|---|---|
| | Post hoc | ANOVA | Study day* |
| | Inulin | Study day | Intervention |
| | (young-older) | Intervention | Study day* group |
| | (post-pre) | group | Intervention* group |
| Whole body | −0.06 | 0.05 | 0.856 |
| production | −0.54, 0.41 | 0.809 | 0.773 |
| μmol/min | P = 1 | 0.596 | 0.002 |
| Size of | −0.2 | 0.141 | 0.377 |
| inaccessible pool, | −0.99, 0.59 | 0.925 | 0.631 |
| (Q1), μmol | P = 0.992 | 0.365 | 0.149 |
| Flux between | −0.14 | 0.083 | 0.614 |
| pools F₂₁ = F₁₂, | −0.19, −0.09 | 0.843 | 0.646 |
| μmol/min | P = <.001 | 0.448 | <.001 |
| Size of | 19.73 | 0.005 | 0.011 |
| inaccessible pool, | −1. 40.46 | 0.331 | 0.005 |
| (Q2), μmol | | 0.004 | 0.75 |
| Production in | 3.94 | 0.004 | 0.019 |
| inaccessible pool | −0.48, 8.35 | 0.381 | 0.24 |
| (U₂ = F₀₂), | P = 0.342 | 0.005 | 0.921 |
| μmol/min | | | |
| Ratio Q₁/Q₂ | −0.002 | 0.758 | 0.015 |
| | −0.003, −0.002 | 0.713 | 0.298 |
| | P = <.001 | <.001 | 0.151 |
| Clearance | 0.041 | 0.209 | 0.165 |
| L/min | −0.316, 0.397 | 0.404 | 0.81 |
| | P = 1 | 0.494 | 0.392 |

Values are estimated marginal means and estimated marginal mean differences between post- and preintervention data with 95% CI. Statistics were performed by generalized linear mixed model with fixed effects:study day (before or after treatment), intervention (placebo or inulin), group (young or older adults), fiber intake and random effects: subject code. The gamma family with Log link was used. Turkey was used to correct the P values for multiple comparisons. Abbreviations: ANOVA, analysis of variance; CI confidence interval; Fᵢⱼ tracee flux from compartment j to i; Qᵢ , tracee pool size in compartment i; U₂, tracee production in compartment 2 (that is, F₀₂).

### TABLES 6A-6C

One-week inulin and placebo intervention-induced changes in acetate (C4) kinetics in young and older adults

| | | | Young |
|---|---|---|---|
| | Young | Young | adults-placebo |
| | adults-placebo | adults-placebo | Delta post v. |
| | Pre-intervention | Post-intervention | pre-intervention |
| Whole body | 0.93 | 1.17 | 0.25 |
| production | (0.64, 1.33) | (0.87, 1.59) | (−0.03, 0.52) |
| μmol/min | | | P = 0.332 |
| Size of | 1.04 | 1.31 | 0.27 |
| inaccessible pool, | (0.71, 1.52) | (0.06, 1.79) | (−0.05, 0.6) |
| (Q1), μmol | | | P = 0.405 |
| Flux between | 0.98 | 1.25 | 0.28 |
| pools F₂₁ = F₁₂, | (0.73, 1.31) | (0.97, 1.62) | (−0.02, 0.57) |
| μmol/min | | | P = 0.31 |
| Size of | 107 | 119 | 12.3 |
| inaccessible pool, | (77, 148) | (87, 163) | (−21.9, 46.5) |
| (Q2), μmol | | | P = 0.962 |
| Production in | 25 | 28 | 3.1 |
| inaccessible pool | (18, 34) | (20, 38) | (−4.8, 10.9) |
| (U₂ = F₀₂), | | | P = 0.947 |
| μmol/min | | | |
| Ratio Q₁/Q₂ | 0.01 | 0.011 | 0.001 |
| | (0.07, 0.014) | (0.008, 0.015) | (−0.003, 0.004) |
| | | | P = 0.9911 |
| Clearance | 0.982 | 1.248 | 0.265 |
| L/min | (0.919, 1.051) | (1.167, 1.335) | (0.241, 0.29) |
| | | | P = <.001 |
| | | | Young |
| | Young | Young | adults-inulin |
| | adults-inulin | adults-inulin | Delta post v. |
| | Pre-intervention | Post-intervention | pre-intervention |
| Whole body | 1.16 | 1.59 | 0.44 |
| production | (0.78, 1.72) | (1.15, 2.22) | (0.07, 0.8) |
| μmol/min | | | P = 0.091 |
| Size of | 1.39 | 1.78 | 0.39 |
| inaccessible pool, | (0.02, 2.1) | (1.26, 2.51) | (−0.05, 0.83) |
| (Q1), μmol | | | P = 0.354 |
| Flux between | 1.24 | 1.67 | 0.43 |
| pools | (0.92, 1.68) | (1.28, 2.19) | (0.04, 0.83) |
| F₂₁ = F₁₂, μmol/min | | | P = 0.151 |
| Size of | 120 | 189 | 69.2 |
| inaccessible pool, | (82, 174) | (132, 271) | (17.8, 120.8) |
| (Q2), μmol | | | P = 0.041 |
| Production in | 28 | 44 | 16.2 |
| inaccessible pool | (19, 41) | (31, 63) | (4.3, 28.1) |
| (U₂ = F₀₂), | | | P = 0.0376 |
| μmol/min | | | |
| Ratio Q₁/Q₂ | 0.012 | 0.01 | −0.002 |
| | (0.008, 0.017) | (0.008, 0.013) | (−0.005, 0002) |
| | | | P = 0.9055 |
| Clearance | 1.095 | 1.174 | 0.079 |
| L/min | (1.024, 1.171) | (1.098, 1.256) | (0.062, 0.097) |
| | | | P = <.001 |

TABLE 6B

| | Older | Older | Older |
|---|---|---|---|
| | adults- | adults- | adults- |
| | placebo | placebo | placebo |
| | Pre- | Post- | Delta post v. |
| | intervention | intervention | pre-intervention |
| Whole body | 1.1 | 1.35 | 0.25 |
| production | (0.84, 1.44) | (1.08, 1.68) | (0, 0.49) |
| μmol/min | | | P = 0.211 |
| Size of | 1.26 | 1.61 | 0.35 |
| inaccessible pool, | (0.95, 1.66) | (1.28, 2.02) | (0.06, 0.65) |
| (Q1), μmol | | | P = 0.96 |
| Flux between | 1.19 | 1.48 | 0.29 |
| pools F₂₁ = _{F12}, | (0.96, 1.47) | (1.22, 1.79) | (0.01, 0.57) |
| μmol/min | | | P = 0.184 |
| Size of | 69 | 70 | 1.3 |
| inaccessible pool, | (54, 87) | (56, 88) | (−14, 16.6) |
| (Q2), μmol | | | P = 1 |
| Production in | 16 | 17 | 0.4 |
| inaccessible pool | (13, 20) | (13, 21) | (−3.2, 4) |
| (U₂ = F₀₂), | | | P = 0.9998 |
| μmol/min | | | |
| Ratio Q₁/Q₂ | 0.019 | 0.023 | 0.004 |
| | (0.014, 0.024) | (0.018, 0.028) | (−9.991, 0.009) |
| | | | P = 0.3722 |
| Clearance | 1.342 | 1.642 | 0.301 |
| L/min | (1.255, 1.435) | (1.536, 1.756) | (0.271, 0.33) |
| | | | P = <.001 |
| | Older | Older | Older |
| | adults-inulin | adults-inulin | adults-inulin |
| | Pre- | Post- | Delta post v. |
| | intervention | intervention | pre-intervention |
| Whole body | 0.96 | 1.27 | 0.32 |
| production | (0.72, 1.27) | (1.01, 1.61) | (0.1, 0.54) |
| μmol/min | | | P = 0.025 |
| Size of | 1.12 | 1.45 | 0.33 |
| inaccessible pool, | (0.84, 1.5) | (1.14, 1.85) | (0.07, 0.58) |
| (Q1), μmol | | | P = 0.072 |
| Flux between | 1.05 | 1.37 | 0.33 |
| pools F₂₁ = F₁₂, | (0.84, 1.3) | (1.13, 1.67) | (0.07, 0.58) |
| μmol/min | | | P = 0.058 |
| Size of | 58 | 84 | 25.8 |
| inaccessible pool, | (45, 76) | (65, 109) | (.1, 42.4) |
| (Q2), μmol | | | P - 0.012 |
| Production in | 14 | 20 | 6.1 |
| inaccessible pool | (11, 18) | (15, 26) | (2.2, 9.9) |
| (U₂ = F₀₂), | | | P = 0.0111 |
| μmol/min | | | |
| Ratio Q₁/Q₂ | 0.018 | 0018 | −0.001 |
| | (0.014, 0.024) | (0.015, 0.022) | (−0.005, 0.004) |
| | | | P = 0.9995 |
| Clearance | 1.238 | 1.279 | 0.04 |
| L/min | (1.158, 1.324) | (1.196, 1.368) | (0.023, 0.06) |
| | | | P = <.001 |

TABLE 6C

| | | | ANOVA |
|---|---|---|---|
| | Post hoc | ANOVA | Study day* |
| | Inulin | Study day | Intervention |
| | (young-older) | Intervention | Study day* group |
| | (post-pre) | group | Intervention* group |
| Whole body | 0.06 | 0.002 | 0.364 |
| production | −0.14, 0.26 | 0.403 | 0.831 |
| μmol/min | P = 0.982 | 0.915 | 0.08 |
| Size of | 0.03 | 0.005 | 0.852 |
| inaccessible pool, | −0.21, 0.27 | 0.408 | 0.926 |
| (Q1), μmol | P = 1 | 0.919 | 0.078 |
| Flux between | 0.05 | 0.003 | 0.502 |
| pools F₂₁ = F₁₂, | −0.16, 0.26 | 0.437 | 0.859 |
| μmol/min | P = 1 | 1 | 0.086 |
| Size of | 21.7 | 0.014 | <.001 |
| inaccessible pool, | −4.7, 48.1 | 0.127 | 0.625 |
| (Q2), μmol | P = 0.43 | <.001 | 0.155 |
| Production in | 5.1 | 0.0121 | <.001 |
| inaccessible pool | −1, 11.2 | 0.1299 | 0.6128 |
| (U₂ = F₀₂), | P = 0.4226 | <.001 | 0.1471 |
| μmol/min | | | |
| Ratio Q₁/Q₂ | −0.001 | 0.0642 | 0.8439 |
| | −0.003, 0.002 | <.001 | 0.0642 |
| | P = 9.9975 | 0.1018 | <.001 |
| Clearance | 0.019 | 0.48 | 0.145 |
| L/min | 0.009, 0.029 | 0.221 | 0.48 |
| | P - 0.002 | 0.069 | 0.221 |

Values are estimated marginal means and estimated marginal mean differences between post- and preintervention data with 95% CI. Statistics were performed by generalized linear mixed model with fixed effects:study day (before or after treatment), intervention (placebo or inulin), group (young or older adults), fiber intake and random effects: subject code. The gamma family with Log link was used. Turkey was used to correct the P values for multiple comparisons. Abbreviations: ANOVA, analysis of variance; CI confidence interval; Fᵢⱼ tracee flux from compartment j to i; Qᵢ , tracee pool size in compartment i; U₂, tracee production in compartment 2 (that is, F₀₂).

### SCFA Concentrations

Preintervention fecal concentrations of acetate, propionate, and butyrate were not different between older and young adults (Table 7). In contrast to fecal concentrations, the preintervention plasma concentrations (μmol/L) of acetate [35 (60.14, 9.86); P=0.011] and butyrate [0.37 (0.64, 0.09); P=0.013] were lower, whereas that of valerate [0.11 (0.06, 0.15); P<0.001] and isobutyrate [0.11 (0.05, 0.16); P=0.001] were higher in older compared with young adults, resulting in a lower BCFA/SCFA ratio [50 (90, 10); P=0.021] (Table 7). Postintervention fecal acetate was higher after inulin compared with placebo supplementation and 2-methylbutyrate lower. No differences between interventions were observed in young or older adults. Only observed increased plasma butyrate concentration after inulin in older adults was observed.

TABLE 7
Baseline short-chain fatty acid fecal and plasma concentrations
in young and older adults

| | | | |
|---|---|---|---|
| Young | | Older adults − | P |
| adults | Older adults | young adults | value |

Preintervention (branched) short-chain fatty acid
concentrations in feces

| | | | | |
|---|---|---|---|---|
| Acetate μmol/g | 216,063 | 186,086 | −29,977 | 0.583 |
| dry weight | (143,230, | (129,879, | (−135,695, | |
| | 325,932) | 266,617) | 75,741) | |
| Propionate | 62,239 | 49,361 | −12,879 | 0.425 |
| μmol/g dry | (40,974, | (33,191, | (−44,081, | |
| weight | 94,541) | 73,406) | 18,323 | |
| Butyrate | 45,279 | 38,547 | 6732 | 0.614 |
| μmol/g dry | (27,541, | (24,809, | (−32,584, | |
| weight | 74,443) | 59,892) | 19,120 | |
| Valerate | 4808 | 3580 | −1228 | 0.328 |
| | (3146, 7347) | (2340, 5478) | (−3645, 1190) | |
| Isobutyrate | 7183 | 6415 | −768 | 0.658 |
| | (4866, 10,603) | (4617, 8913) | (−4128, 2592) | |
| Isovalerate | 9678 | 8764 | −914 | 0.691 |
| μmol/g dry | (6601, 14188) | (6374, 12,050) | (−5372, 3544) | |
| weight | | | | |
| 2- | 4337 | 4019 | −318 | 0.755 |
| Methylbutyrate | (2979, 6314) | (2937, 5500) | (−2297, 1661) | |
| μmol/g dry | | | | |
| weight | | | | |
| Fecal BCFA/ | 18.68 | 21.57 | 2.89 | 0.800 |
| SCFA | (6.93, 50.38) | (11.14, 41.78) | (−19.2, 24.07) | |
| Fecal dry | 0.29 | 0.28 | −9.01 | 0.638 |
| Matter (%) | (0.25, 0.34) | (0.25, 0.31) | (−0.06, 0.04) | |

Preintervention (branched) short-chain fatty acid concentrations in
systemic circulation

| | | | | |
|---|---|---|---|---|
| Acetate μmol/L | 99.35 | 64.35 | −35 | 0.011 |
| | (79.7, 123.69) | (50.13, 82.62) | (−60.14, −9.86) | |
| Propionate | 1.37 | 1.56 | 0.2 | 0.214 |
| μmol/ | (1.14, 1.64) | (1.38, 1.77) | (−0.11, 0.5) | |
| Butyrate, μmol/ | 1.13 | 0.76 | −0.37 | 0.013 |
| | (0.91, 1.4) | (0.6, 0.96) | (−0.64, −0.09) | |
| Valerate, μmol/ | 0 | 0.1 | 0.11 | <.001 |
| | (−0.04, 0.03 | (0.08, 0.13) | (0.06, 0.15) | |
| Isobutyrate | 0.15 | 0.25 | 0.11 | 0.001 |
| μmol/ | (0.11, 0.2) | (0.22, 0.29) | (0.05, 0.16) | |
| Isovalerate | 0.85 | 0.85 | 0 | 0.988 |
| μmol/ | (0.67, 1.07) | (0.71, 1.01) | (−0.24, 0.24) | |
| 2- | 0.074 | 0.107 | 0.033 | 0.073 |
| Methylbutyrate | (0.05, 0.11) | (0.086, 0.133) | (−0.002, 0.068) | |
| μmol/ | | | | |
| Plasma BCFA/ | 112 | 62 | −50 | 0.021 |
| SCFA (ratio) | (83, 152) | (41, 93) | (−90, −10) | |

Values are estimated marginal means (95% CI). Statistics were performed by generalized linear model with family: gamma and link: Log. Covariants included biological sex and fiber intake.
Abbreviations: BCFA, branched short-chain fatty acid; CI, confidence interval; SCFA, short-chain fatty acid.

### Associations Between SCFA Production and Concentrations

To test whether SCFA concentrations are adequate estimates of WBP and production in the inaccessible pool (U2. F₀₂), preintervention WBP and U2 was correlated with plasma and fecal concentrations on a whole-group level. Positive relationships were found between WBP and U2 with plasma concentration of acetate, propionate and butyrate. In contrast, no relationships were observed with fecal concentrations.

### DISCUSSION

In this randomized, double-blind, placebo-controlled, crossover study, it was observed that a one week supplementation of the fermentable fiber inulin (final dose: 30 g/d) mainly increased butyrate pool sizes (Q2) and production rates in the inaccessible pool (U2. F02; that is, the pool SCFAs drain into after intestinal microbial production and after endogenous production in other organs (FIG. 4). Compared with young adults (age range: 20-29 y), older adults (age range: 59-87 y) had lower preintervention SCFA production rates, pool sizes and fluxes butinulin supplementation evoked a similar increase in SCFA kinetics parameters. These observations were made using a novel stable tracer SCFA pulse approach in combination with compartmental modeling (4 \). As reasonable relationships were found between plasma concentrations of SCFAs with production in the inaccessible pool, measuring plasma SCFAs may be used as a proxy of SCFA production.

### Side Effects and Compliance

Inulin was chosen as a fiber supplement as it is efficiently fermented with a high yield of SCFAs and therefore is commonly used in intervention studies (6 \). Participants experienced more overall gastrointestinal symptoms during inulin intervention indicating gas formation caused by successful inulin fermentation. Both groups had an elevated feeling of indigestion, representing more frequent borborygmus, abdominal distension, eructation, and increased flatus. Young adults additionally had higher diarrhea and abdominal pain subscore (7 \). Despite side effects mainly being reported in young adults, the only postintervention difference observed for indirect measures of intestinal transit time was a lower relative amount of fecal dry weight in the older adults (8 \) whereas Bristol Stool Scale score did not change in either group. Previous research did not report changes in transit time after inulin intake (9, 10). Side effects did not lead to higher mood disturbance scores during inulin supplementation and compliance of the participants was high with overall 98% of placebo doses and 97% of inulin doses consumed.

A high final supplement dose (30 g/d) above recommended intervention dosage was selected to ensure a measurable effect on SCFA production and kinetics. Supplement doses were increased from 10 g/d to 30 g/d throughout the week for acclimation of the colon and microbiome to the increased fiber intake, and split into a morning and evening dose to increase tolerability and to ensure a steady arrival of fiber in the colon.

### SCFA Metabolism

Despite a higher habitual dietary fiber intake, preintervention parameters of SCFA metabolism were lower in older than young adults, which is comparable with our previous results for most of the calculated pool sizes and fluxes (4). In the current study, preintervention acetate and butyrate WBP and production in the inaccessible pool (U2. F₀₂) as well as acetate and butyrate Q2 pool sizes were lower in older adults. The whole-body rate of appearance for acetate, propionate, and butyrate (871.8, 17.8, and 18.4 μmol/min, respectively) previously measured in young adults using a primed, constant continuous stable tracer SCFA infusion (11) were 5.1-fold (acetate), 5.7-fold (propionate), and 17.9-fold (butyrate) higher than the mean WBP measured in the young adults in this study. However, the molar ratio was with 96:2:2, relatively comparable with the instant ratio of 97:2:1. These differences might have mainly been caused by insufficient priming of the tracer pool in the constant, continuous infusion protocol resulting in an overestimation of WbRa.

Interestingly, the relation between the SCFA production, measured with the WBP does not represent the differences between young and older adults as measured by the production in the inaccessible pool. Therefore, this indicates that measuring the WBP, as is done with a primed-constant infusion protocol (11), cannot be used to estimate the production of SCFAs in the inaccessible pool. Similarly, preintervention fecal concentrations of the straight-chain SCFAs acetate, propionate, butyrate, and fecal BCFA/SCFA ratio were not lower in the older adults. In contrast, others have found that aging is related to higher fecal BCFA/SCFA ratios (12) and reduced SCFA concentrations (12 13) despite a maintained absolute number of SCFA-producing bacteria (13). In contrast, lower plasma concentrations of acetate and butyrate and a lower BCFA/SCFA ratio in older adults was observed. Despite the findings of reduced SCFA metabolism parameters, no differences in fecal concentrations or relation between these parameters were found. These results agree with others that found that fecal SCFA concentrations inadequately represent SCFA concentrations or production in the proximal colon (14) due to efficient SCFA absorption by colonocytes (15, 16). Fecal SCFA concentrations have now been proposed as a marker for distal SCFA production and/or absorption (15). In contrast to fecal concentrations, plasma concentrations of valerate and of the BCFAs isobutyrate and 2-methylbutyrate were higher in older adults potentially due to increased proteolytic fermentation (17) in the proximal colon or reduced disposal from the systemic circulation (3).

In summary, the reduced preintervention production of beneficial SCFAs in combination with increased systemic BCFA concentrations, a marker of proteolytic fermentation with harmful byproducts (18) could potentially be explained by the previously suggested switch from saccharolytic to proteolytic fermentation in aging (17).

### Postintervenion Metabolism

A 1-wk supplementation with the fermentable fiber inulin increased several parameters of acetate and butyrate production and pool sizes in young and older adults with no clear differences between young and older adults after correction for habitual dietary fiber intake. Compartmental parameters of propionate only showed an increase in Q2 and production in the inaccessible pool. It is believed that SCFA release from inulin fermentation is in the order of acetate>>butyrate>propionate (19,).

It was determined whether changes in butyrate production measured herein using the pulse approach and compartmental modeling match previously reported results to validate the instant findings. Butyrate was selected as it is less influenced by systemic, nonmicrobial production than acetate. The increase in young participants was equivalent to 23.3 mmol/d and in older participants to 8.7 mmol/d, although this was not statistically different. It was previously estimated that 1 g of inulin fermentation yields 1.31 mmol butyrate in young adults under assumption of a 5% butyrate bioavailability, which is equivalent to 39.3 mmol for 30 g inulin (11). Although a statistical difference between young and older adults could not be observed, it is believed the lower production in older adults at baseline in relation to fiber intake and the difference in the response to the inulin intervention suggests that older adults have a lower capability to increase SCFA production on fiber. Post inulin intervention production (U2) of acetate did not increase. Acetate production in plasma has several sources, besides from activity in the microbiome. For instance, acetate production is also from ketogenesis in the liver (20, 21). Therefore, it is possible that an increase in acetate production after inulin intervention was not able to be detected.

In contrast to the production results, acetate concentration in fecal dry weight was higher after inulin compared with placebo supplementation, mainly in older adults. Previous studies did not detect fecal SCFA concentration changes after a variety of dosages and durations of inulin supplementation (20) despite complete fermentation (22). These results question the suitability of fecal concentration measurements as estimates for SCFA production (15, 16). Soluble fibers like inulin are mainly fermented in the proximal colon (22) and efficiently absorbed (16). Inulin might have been less efficiently fermented in the older adults, as implied by the lower increase in SCFA U2, and thus more inulin might have been available for fermentation in the distal colon increasing fecal acetate concentrations in older adults. Increased fecal concentrations may also be an indicator for insufficient SCFA absorption by colonocytes (16). Fiber intake, in particular of less fermentable fibers, can shorten intestinal transit time, as potentially observed in our older adult group based on the decreased fecal dry weight ratio. This in turn elevates the availability of fiber for microbial fermentation in the distal colon and reduces the time for SCFA absorption (15, 24). Therefore, fiber intake might increase fecal SCFA concentrations independent of SCFA production through alteration of transit time and absorption rates or not alter fecal SCFA concentrations if fiber is efficiently fermented and absorbed before excretion with stool (22).

In conclusion, 1-wk, high-dose inulin supplementation increases SCFA production in individuals of different ages. Overall increase in SCFA production was moderate considering the high final inulin supplement dose administered (30 g/d). It is contemplated that approaches reliably increasing SCFA production without inducing gastrointestinal symptoms and investigate related systemic effects. Plasma but not fecal concentrations correlated with SCFA production and should be used to estimate SCFA production when stable tracer methods cannot be used.

### REFERENCES

1. Food and Drug Administration (FDA), Food labeling and nutrition: Questions and Answers on Dietary Fiber, 2021. fda.gov/food/food-labeling-nutrition/questions-and-answers-dietary-fiber #fda_actions.
2. Food and Drug Administration (FDA), Daily Value and Percent Daily Value: Changes on the New Nutrition and Supplement Facts Labels.
3 Deutz, et al., Metabolism 78:167-178, 2018.
4. Kirschner, et al., Metabolism, 141:155399, 2023.
5. JASP Team, 2024. jasp-stats.org/.
6. Szczesniak, et al. Nutr. Neurosci. 19 (7): 279-283, 201.6
7. Svedlund, et al., Dig. Dis. Sci. 33 (2), 129-134, 1988.
8. Vork, et al, The Korean Society of Neurogastroenterology and Motility; 25:625, 2019 Jan. 14.
9. J. Slavin and J. Feirtag, Food Funct, 2 (1): 72-77, 2011.
10. Holmes, et al., Microbiome 10 (1): 114, 2022.
11. Salazar, et al., J. Am. Coll. Nutr. 32 (6): 399-406, 2013.
12. Salazar, et al., Nutrients 11 (8): 1765, 2019.
13. S. Bischoff, Curr. Opin. Clin. Nutr. Metab. Care. 19 (1): 26-30, 2016.
14. Cummings, et al., Gut, 28 (10): 1221-1227, 1987.
15. Ghosh, et al., Nat. Rev. Gastroenterol. Hepatol. 19 (9): 565-584, 2022.
16. Moorefield, et al., Aging 9 (8) (2017) 1898-1915.
17. Kirschner, et al., Curr. Opin. Clin. Nutr. Metab. Care. 24 (5): 464-472, 2021.
18. Boets, et al., Nutrients 7 (11) (2015) 8916-8929, 2015.
19. Deroover, et al., Nutrients 9 (1): 83, 2017.
20. Yamashita, et al., Biochim Biophys Acta, 1532:79-87, 2001.
21. Knowles, et al., Biochem J, 142:401-11, 1974.
22. Alles, et al., Br. J. Nutr., 76 (2): 211-221, 1996.
23. Klurfeld, et al., Am. J. Physiol. Endocrinol. Metab. 315 (6): E1087-E1097, 2018.
24. Müller, et al., Am. J. Physiol. Gastrointest. Liver Physiol. 318 (2): G361-G369, 2020.
25. Holscher, H. D., Gut Microbes 8 (2): 172-184, 2017.
26. Kimura, et al., Physiol Rev, 100:171-210, 2020.
27. van der Hee, Trends Microbiol 29 (8) (2021) 700-712.
28. E. A. Smith, G. T. Macfarlane, Anaerobe 3 (5) (1997) 327-337.
29. Macfarlane, et al., FEMS Microbiol. Ecol. 101 (2) (1992) 81-88.
30. Szczesniak, et al., Nutr. Neurosci. 19 (7): 279-283, 2016.
31. Granado-Serrano, et al., Sci. Rep. 9 (1): 1772, 2019.
32. Salazar, et al., J. Am. Coll. Nutr. 32: (6): 399-406, 2013.
33. Bischoff, S., Curr. Opin. Clin. Nutr. Metab. Care. 19 (1): 26-30, 2016.
34. Grosicki, et al., Calcif. Tissue Int. 102 (4): 433-442, 2018.
35. Ghosh, et al., Nat. Rev. Gastroenterol. Hepatol. 19 (9): 565-584, 2022.
36. Moorefield, et al., Aging 9 (8): 1898-1915, 2017.
37. J. H. Cummings and G. T. Macfarlane, J. Appl. Bacteriol. 70 (6): 443-459, 1991.
38. Yasuda, et al., J Nutr, 137:2399-404, 2007.
39. Olano-Martin, et al., Br. J. Nutr., 83 (3): 247-255, 2000.
40. Almutairi, et al., Eur. J. Nutr., 61 (6): 2853-2871, 2022.

What is claimed is:

1. A method for diagnosing chronic obstructive pulmonary disease (COPD) in a subject, comprising:
a) calculating a metabolic rate of at least one stable-isotope labeled short-chain fatty acid administered with a soluble fiber to the subject and to a healthy control, comprising:
i) drawing, after fasting, a baseline first blood sample from the subject and from the healthy control;
ii) administering intravenously to the subject and to the healthy control at least one stable isotope labeled short-chain fatty acid;
iii) drawing a series of second blood samples at intervals from the subject and from the healthy control;
iv) measuring a concentration of the isotope in the first blood sample and in each of the series of second blood samples from the subject and from the healthy control;
v) applying a compartmental analysis to calculate a first metabolic rate of the at least one stable isotope labeled short-chain fatty acid from the concentrations of the isotope for the subject and for the healthy control;
vi) administering orally a soluble fiber to the subject and to the healthy control; and
vii) repeating steps ii) to v) to calculate a second metabolic rate for the at least one stable isotope labeled short-chain fatty acid for the subject and for the healthy control; and
b) comparing the metabolic rate in the subject to the metabolic rate in the healthy control, wherein a lower metabolic rate in the presence of soluble fiber in the subject indicates a deficiency in production of short-chained fatty acids, thereby diagnosing the pulmonary disease.

2. The method of claim 1, wherein step iii) comprises drawing the series of second blood samples at intervals of about 5 minutes to about 20 minutes over a period of about 1 hour to about 5 hours.

3. The method of claim 1, wherein an interval between step vi) and step vii) is about 1 day to about 7 days.

4. The method of claim 1, wherein the stable isotope labeled short-chain fatty acid is ¹³C-acetate, ¹³C-propionate, ¹³C-butyrate, ¹³C-iso-butyrate, ¹³C-valerate, or ¹³C-iso-valerate.

5. The method of claim 1, wherein the soluble fiber is inulin, oligofructose or fructooligosaccharide or a combination thereof.

6. A method for diagnosing the presence of autism in a subject, comprising:
a) calculating a metabolic rate of at least one short-chain fatty acid, at least one amino acid and at least one protein in the subject and in a healthy control;
i) drawing, after fasting, a baseline first blood sample from the subject and from the healthy control;
ii) administering at intervals to the subject and to the healthy control at least one stable first isotope labeled short-chain fatty acid intravenously; at least one amino acid orally; at least one stable second isotope labeled amino acid intravenously;
and a liquid nutrition formula comprising at least one stable third isotope labeled protein orally;
iii) drawing a series of second blood samples at intervals from the subject and from the healthy control;
iv) measuring a concentration of each of the stable first isotope, the stable second isotope and the stable third isotope in the first blood sample and in each of the series of second blood samples from the subject and from the healthy control; and
v) applying a compartmental analysis to calculate the metabolic rate of the at least one short-chain fatty acid, the at least one amino acid and the at least one protein in the subject and in a healthy control; and
b) comparing each of the metabolic rates in the subject to each of the metabolic rates in the healthy control; wherein a decrease in the metabolic rate of the at least one of the stable isotope labeled short-chain fatty acid, of the at least one stable second isotope labeled amino acid or of the at least one stable third isotope labeled protein or a combination thereof in the subject indicates a deficiency in production of short-chained fatty acids and quality of digestion, thereby diagnosing the neurological disorder.

7. The method of claim 6, wherein in step ii) administering the at least one stable first isotope is by pulse and the at least one stable second isotope is by primed continuous infusion.

8. The method of claim 6, wherein in step ii) administering the liquid nutrition formula is performed periodically in intervals between about every 10 minutes and about every 30 minutes for about 2 hours to about 4 hours.

9. The method of claim 6, wherein step iii) comprises drawing the series of second blood samples at intervals of about 5 minutes to about 20 minutes over a period of about 1 hour to about 5 hours.

10. The method of claim 6, wherein step v) comprises applying a compartmental analysis to calculate the metabolic rate.

11. The method of claim 6, wherein the stable isotope labeled short-chain fatty acid is ¹³C-acetate, ¹³C-propionate, ¹³C-butyrate, ¹³C-iso-butyrate, ¹³C-valerate, or ¹³C-iso-valerate.

12. The method of claim 6, wherein the amino acid is L-allo-a isoleucine, phenylalanine, tyrosine, leucine, tryptophan, or valine or a combination thereof.

13. The method of claim 6, wherein the stable second isotope labeled amino acid is a ¹⁵N-L-allo-¹⁵N-isoleucine, a ¹⁵N-phenylalanine, a ¹⁵N-tyrosine, a ¹⁵N-leucine, a ¹⁵N-tryptophan, or a ¹⁵N-valine.

14. The method of claim 6, wherein the at least one stable third isotope labeled protein is a 15N-labeled protein.

15. A method for increasing short chain fatty acid production in an older subject, comprising:
a) measuring a concentration of at least one short chain fatty acid in the older subject;
b) supplementing the subject's diet with inulin over a period of time; and
c) measuring the concentration of the at least one short chain fatty acid after supplementing to confirm an increase thereof in the older subject.

16. The method of claim 15, further comprising repeating steps b) and c) at least once.

17. The method of claim 15, wherein steps a) to c) comprise:
drawing a baseline first blood sample from the older subject after fasting to determine background concentrations of the short chain fatty acids;
adding an amount of inulin to the subject's diet over the period of time;
administering to the older subject ¹³C-labeled short chain fatty acids;
drawing a series of second blood samples at intervals from the older subject; and
applying a compartmental analysis on the series of second blood samples to determine concentrations of the ¹³C-labeled short chain fatty acids; wherein concentrations of the ¹³C-labeled short chain fatty acids greater than the background concentrations of the short chain fatty acids indicates an increase in the short chain fatty acid production in the older adult.

18. The method of claim 15, wherein the short chain fatty acids are acetate, propionate and butyrate.

19. The method of claim 15, wherein the period of time is about 7 days.

20. The method of claim 19, wherein the inulin supplements the subject's diet in increasing amounts over the period of the seven days.
